(19) United States
(12) Patent Application Publication
(10) Pub. No.: US 2025/0201002 A1
(43) Pub. Date: Jun. 19, 2025

(54) SYSTEMS AND METHODS FOR IMAGE-ACTIVATED PARTICLE SORTING BASED ON AI GATING

(71) Applicant: The Regents of the University of California, Oakland, CA (US)

(72) Inventors: Yu-Hwa LO, San Diego, CA (US); Rui TANG, San Diego, CA (US)

(21) Appl. No.: 18/960,318

(22) Filed: Nov. 26, 2024

Related U.S. Application Data
(63) Continuation of application No. PCT/US2023/067943, filed on Jun. 5, 2023.
(60) Provisional application No. 63/365,836, filed on Jun. 3, 2022.

(51) Int. Cl. G06V 20/69; G01N 15/1434; G06V 10/141; G06V 10/82

(52) U.S. Cl. CPC G06V 20/693; G01N 15/1436; G06V 10/141; G06V 10/82; G06V 20/698

(57) ABSTRACT

Disclosed are systems, devices and methods for imaging and image-activated sorting of particles in a flow system based on AI gating. In some aspects, a system includes a particle flow device to flow particles through a channel, an imaging system to obtain image data of a particle during flow through the channel, and a control command unit to produce a control command for sorting the particle based on an AI-based gating model and the image data, and an actuator to direct, according to the control command, the particle into one of a plurality of output paths of the particle flow device in real-time.

### CROSS-REFERENCE

This patent document is a continuation of International Application No. PCT/US2023/067943, filed Jun. 5, 2023, which claims priority to and benefits of U.S. Provisional Patent Application No. 63/365,836, entitled “IMAGE-ACTIVATED CELL SORTING USING DEEP LEARNING AND AI INFERENCING” filed on Jun. 3, 2022. The entire content of the aforementioned patent application is incorporated by reference as part of the disclosure of this patent document.

### STATEMENT REGARDING FEDERALLY SPONSORED RESEARCH OR DEVELOPMENT

This invention was made with government support under grant no. 2R44DA045460-02 awarded by the National Institutes of Health (NIH). The government has certain rights in the invention.

### TECHNICAL FIELD

This patent document relates to systems, devices and techniques for particle sorting, and in particular low-latency image-activated particle sorting based on AI gating.

### BACKGROUND

Flow cytometry is a technique to detect and analyze particles, such as living cells, as they flow through a fluid. For example, a flow cytometer device can be used to characterize physical and biochemical properties of cells and/or biochemical molecules or molecule clusters based on their optical, electrical, acoustic, and/or magnetic responses as they are interrogated in a serial manner. Typically, flow cytometry uses an external light source to interrogate the particles, from which optical signals are detected caused by one or more interactions between the input light and the particles, such as forward scattering, side scattering, and fluorescence. Properties measured by flow cytometry include a particle's relative size, granularity, and/or fluorescence intensity.

Particle sorting, including cell sorting at the single-cell level, has become an important feature in the field of flow cytometry as researchers and clinicians become more interested in studying and purifying certain cells, e.g., such as stem cells, circulating tumor cells, and rare bacteria species.

### SUMMARY

The technology disclosed in this document can be implemented to provide methods, devices and systems for producing images of particles in a flow system, and in specific configurations, the disclosed technology can be used for imaging particles in real time and subsequently sorting particles, including cells, based on a trained gating model and image data of individual particles. The disclosed techniques can be applied for producing cell images and sorting cells in flow cytometers in real time. In applications, the disclosed technology can be used to detect and sort cells based on the bright field signals, fluorescent signals and/or scattering intensity.

In implementations, for example, the disclosed systems possess the high throughput of flow cytometers and high spatial resolution of imaging cytometers, in which the particle images are produced at a fast enough rate to accommodate real-time particle sorting in a flow system based on machine ascertainable physical and/or physiological properties of the particle represented in and image data and analyzed using an AI based gating model.

In some embodiments in accordance with the present technology, a particle flow device including a substrate, a channel formed on the substrate operable to allow individual particles to flow along a flow direction to a first region of the channel, and two or more output paths branching from the channel at a second region proximate to the first region in the channel, an imaging system interfaced with the particle flow device and operable to obtain image data associated with a particle when the particle is flowing in the first region through the channel, a control command unit including a processor configured to produce a control command indicative of a particle class determined based on a gating model and the image data; and an actuator operatively coupled to the particle flow device and in communication with the control command unit, the actuator operable to direct the particle into an output path of the two or more output paths based on the control command, wherein the image-activated particle sorting system is operable to sort the individual particles during flow in the channel.

In some embodiments in accordance with the present technology, a method for image-based sorting of a particle includes obtaining, by an imaging system interfaced with a particle flow device, image data of a particle flowing through a channel of the particle flow device; producing, by a control command unit, a control command indicative of a particle class of the particle determined based on a gating model and the image data; and directing the particle into one of a plurality of output paths of the particle flow device based on the control command.

The above and other aspects of the disclosed technology and their implementations and applications are described in greater detail in the drawings, the description and the claims.

### BRIEF DESCRIPTION OF THE DRAWINGS

FIG. 1A shows a diagram of an example embodiment of an image-activated particle sorting system in accordance with the disclosed technology.

FIG. 1B shows a block diagram of an example control command unit of an image-activated particle sorting system in accordance with embodiments of the present document.

FIG. 1C shows a diagram of an example process for image-activated particle sorting based on AI gating in accordance with embodiments of the present document.

FIGS. 2A-2C show diagrams of an example image-activated particle sorting microfluidic system in accordance with embodiments of the present document.

FIG. 3A shows an example real-time data processing system architecture in accordance with embodiments of the present document.

FIG. 3B shows an example real-time data processing pipeline in accordance with embodiments of the present document.

FIG. 3C illustrates example beads and cell images captured by an example low-latency IACS system in accordance with embodiments of the present document.

FIG. 4 illustrates optical components of an example low-latency IACS system in accordance with embodiments of the present document.

FIG. 5 illustrates an optical performance measurement of an example low-latency IACS system in accordance with embodiments of the present document.

FIG. 6 illustrates an example detection optics resolution limit measurement with resolution target under scanning laser illumination in accordance with embodiments of the present document.

FIG. 7 illustrates an exemplary architecture of the 2D UNet in accordance with embodiments of the present document.

FIG. 8 shows custom UNet model optimization on model size, training time, and inference time in accordance with embodiments of the present document.

FIG. 9 shows UNet training curves and UNet Inference time with initial convolutional kernel size being 4 in accordance with embodiments of the present document.

FIG. 10 shows UNet training curves and UNet Inference time with initial convolutional kernel size being 8 in accordance with embodiments of the present document.

FIG. 11 shows UNet training curves and UNet Inference time with initial convolutional kernel size being 16 in accordance with embodiments of the present document.

FIG. 12 shows UNet training curves and UNet Inference time with initial convolutional kernel size being 32 in accordance with embodiments of the present document.

FIG. 13 shows UNet training curves and UNet Inference time with initial convolutional kernel size being 64 in accordance with embodiments of the present document.

FIG. 14 shows bead sorting results for beads of 7 μm and 15 μm in accordance with embodiments of the present document.

FIG. 15 shows UNet training curves for beads sorting experiments in accordance with embodiments of the present document.

FIG. 16 shows beads sorting experiment pre-sorting and post-sorting Accuri particle composition analysis in accordance with embodiments of the present document.

FIG. 17 shows human white blood cell sorting results in accordance with embodiments of the present document.

FIG. 18 shows UNet training curves for human white blood cell sorting experiment.

FIG. 19 presents the Accuri particle composition analysis performed before the lymphocyte sorting experiment, providing information about the composition of the initial lymphocyte sample in accordance with embodiments of the present document.

FIG. 20 shows the Accuri particle composition analysis for the post-sorting batch 1 solution in the lymphocyte sorting experiment, offering insights into the composition of the sorted lymphocytes in accordance with embodiments of the present document.

FIG. 21 shows the Accuri particle composition analysis for the post-sorting batch 2 solution in the lymphocyte sorting experiment, providing information about the composition of the sorted lymphocytes in accordance with embodiments of the present document.

FIG. 22 shows the Accuri particle composition analysis for the post-sorting batch 3 solution in the lymphocyte sorting experiment, offering insights into the composition of the sorted lymphocytes in accordance with embodiments of the present document.

FIG. 23 shows the Accuri particle composition analysis performed before the monocyte sorting experiment, providing information about the composition of the initial monocyte sample in accordance with embodiments of the present document.

FIG. 24 presents the Accuri particle composition analysis for the post-sorting batch 1 solution in the monocyte sorting experiment, offering insights into the composition of the sorted monocytes in accordance with embodiments of the present document.

FIG. 25 shows the Accuri particle composition analysis for the post-sorting batch 2 solution in the monocyte sorting experiment, providing information about the composition of the sorted monocytes in accordance with embodiments of the present document.

FIG. 26 shows the Accuri particle composition analysis for the post-sorting batch 3 solution in the monocyte sorting experiment, offering insights into the composition of the sorted monocytes in accordance with embodiments of the present document.

FIG. 27 shows the Accuri particle composition analysis performed before the granulocyte sorting experiment, providing information about the composition of the initial granulocyte sample in accordance with embodiments of the present document.

FIG. 28 presents the Accuri particle composition analysis for the post-sorting batch 1 solution in the granulocyte sorting experiment, offering insights into the composition of the sorted granulocytes in accordance with embodiments of the present document.

FIG. 29 shows the Accuri particle composition analysis for the post-sorting batch 2 solution in the granulocyte sorting experiment, providing information about the composition of the sorted granulocytes in accordance with embodiments of the present document.

FIG. 30 shows the Accuri particle composition analysis for the post-sorting batch 3 solution in the granulocyte sorting experiment, offering insights into the composition of the sorted granulocytes in accordance with embodiments of the present document.

FIG. 31 shows fluorescence microscopy images of the pre-sorting, post-sorting, and waste beads mixture in accordance with embodiments of the present document.

### DETAILED DESCRIPTION

Image-based detection, classification, and sorting of target cells among a heterogenous cell population can bring phenomenal insight to biomedical research and application. Existing fluorescent-activated cell sorting (FACS) technology optically interrogates individual cells in a single-cell flow stream and isolates cells based on scattering and fluorescence intensity features of the interrogated cells. In comparison, image-activated cell sorting (IACS) systems can classify and sort cells based on spatial features obtained from cell images, which offer much greater information content than existing FACS that is limited to a single value per parameter. By extracting the spatial and morphological features carried by light transmission, scattering, and fluorescent properties of cells, IACS can classify and isolate the targeted cell types from a heterogeneous cell population using image-feature based gating (e.g., cellular size and shape, nuclear size, and shape, nucleus-to-cytoplasm ratio, DNA and RNA localization, cellular organelle localization, cellular aggregation, as well as non-intuitive features). The emergence of the image-activated cell sorting (IACS) technique provides a powerful biomedical research tool for studies of cell cycle, cell-cell interaction, protein localization, DNA and RNA localization, and the relationship between cellular phenotype to genotype. As used herein, IACS may also refer to image-activated particle sorting.

An existing IACS system may be configured to perform real-time data processing and sorting actuation to process high-content image data at a high data transfer rate and extract many image-related features based on which sorting decisions are made. The computing power of the processor of such an IACS system may limit the number of cell image features that can be extracted in real-time as many image-related features cause heavy computation. For example, cell phenotypical and morphological features can be complex and convoluted, not resolvable or correctly identifiable by human vision or some subjective criteria, partly because humans can only process a very small set of images out of a very large sample size. As a result, mathematical representations of image features driven by human-vision-based gating can have deficiencies and miss important biological insight. Additionally or alternatively, although latency of an IACS system may be improved by improving the hardware including, e.g., increasing the number and/or computing power of processors used in image data processing, improving camera-based optics design and hardware, etc., such solutions may suffer from limitations including, e.g., limited scalability due to cost and complexity, sensitivity and motion blur issues in the imaging process, or the like.

To address these and other technical problems, disclosed in this document include systems and methods for image-activated particle sorting employing an artificial intelligence (AI) based gating model with real-time AI inferencing capacity. Based on a deep learning algorithm and artificial intelligence (AI) computing hardware, convolutional neural networks (CNN) can solve complex image-driven pattern recognition problems that are human-vision uninterpretable. Some embodiments of the present documents provide measures for improving the AI-based gating model including, e.g., employing a suitable CNN model (e.g., a UNet CNN autoencoder model), optimizing a model parameter (e.g., identifying a kernel count of the initial convolutional kernels of the CNN model so as to comprehensively optimize training and performance including reducing the training time and/or sorting decision time, while maintaining a sorting accuracy), improving the training process by identifying image features for labelling images to be used as training data. According to some embodiments, a real-time sorting by AI inference with millisecond latency may be achieved using an example image-activated particle sorting system that includes one field-programmable gate array (FPGA) processor for image processing and a Personal Computer (PC) with a dedicated Graphics Processing Unit (GPU) for conducting real-time AI model inference based on an optimized UNet CNN autoencoder model.

In applications, the disclosed technology can be implemented in specific ways in the form of methods, systems, and devices for image-activated cell sorting in flow cytometry using (a) real-time image acquisition of fast travelling particles by efficient data processing techniques utilizing mathematical algorithms implemented with, e.g., FPGA and/or GPU, and concurrently (b) AI based “gating” techniques to make particle classification or sorting decisions based on such real-time acquired particle images as input. Unlike existing flow cytometers that use fluorescent intensities of chosen biomarkers as criteria for cell sorting, the methods, systems, and devices in accordance with the disclosed technology allow for various user-defined gating criteria for sorting labelled particles and also label-free particles in real time.

In some embodiments, an image-activated particle sorting system includes a particle flow device, such as a flow cell or a microfluidic device, integrated with a particle sorting actuator; a high-speed and high-sensitivity optical imaging system; and a real-time particle image processing and sorting control electronic system. For example, an objective of the disclosed methods, systems and devices is to perform the entire process of (i) image capture of a particle (e.g., cell), (ii) image reconstruction from a time-domain signal, and (iii) making a particle sorting decision and sorting operation by the actuator within a latency of less than 15 milliseconds to fulfill the needs for real-time particle sorting. In some implementations described herein, the total latency is less than 5 milliseconds (e.g., 3 milliseconds).

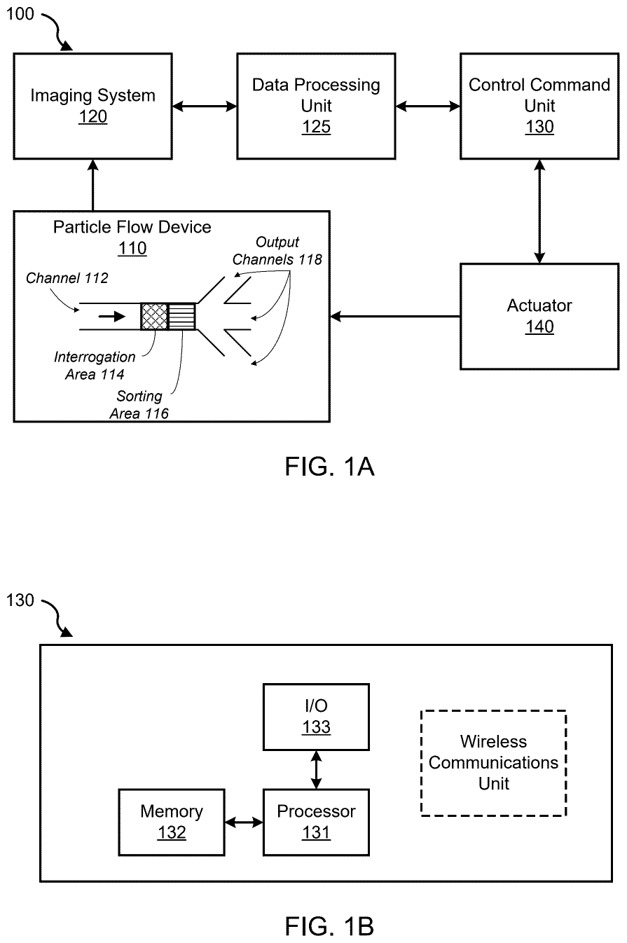

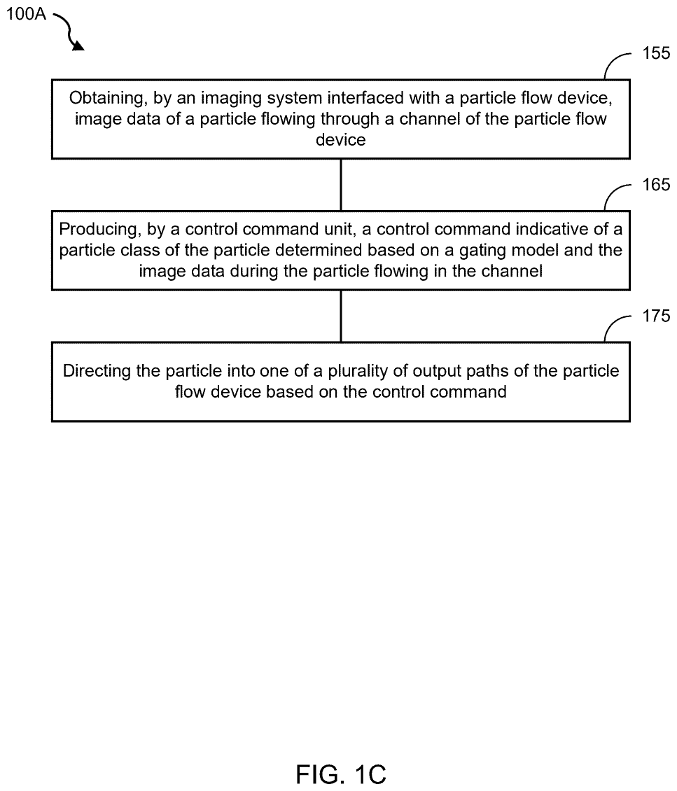

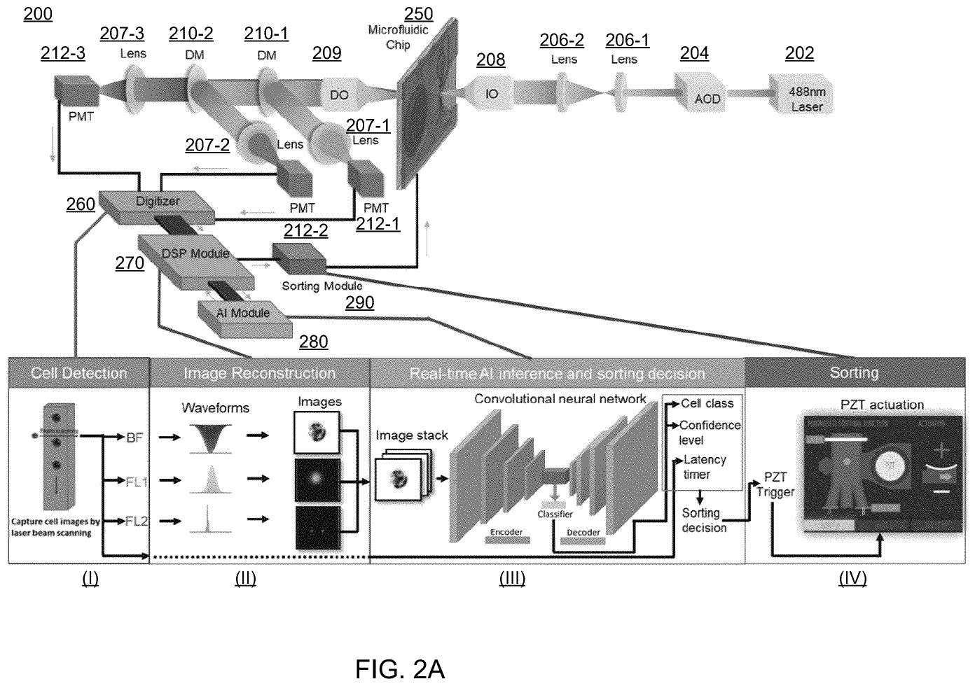

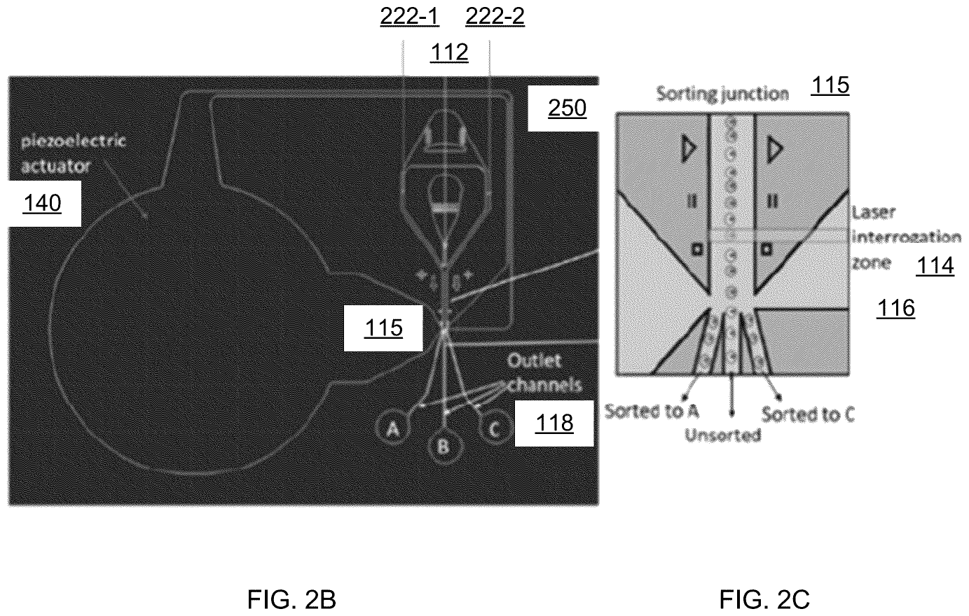

FIG. 1A shows a diagram of an example embodiment of an image-activated particle sorting system 100 in accordance with the present technology. The system 100 includes a particle flow device 110, an imaging system 120 interfaced with the particle flow device 110, a data processing unit 125 in communication with the imaging system 120, a control command unit 130 in communication with the data processing unit 125, and an actuator 140 in communication with the control command unit 130 and operatively coupled to the particle flow device 110. The particle flow device 110 is structured to include a channel 112 in which particles flow along a flow direction to an interrogation area 114 where image data are obtained by the imaging system 120 for each particle in the interrogation area 114. The data processing and control unit 130 is configured to process the image data and determine one or more properties associated with the particle to produce a control command for sorting of the particle. The control command is provided to the actuator 140, which is interfaced with the particle flow device 110 at a sorting area 116 of the device 110, such that the actuator operates to sort the particular particle into an output channel 118 corresponding to the control command. More descriptions regarding the particle flow device 110 may be found elsewhere in the present disclosure. See, e.g., a microfluidic chip 250 as illustrated in FIGS. 2A-2C and relevant descriptions thereof.

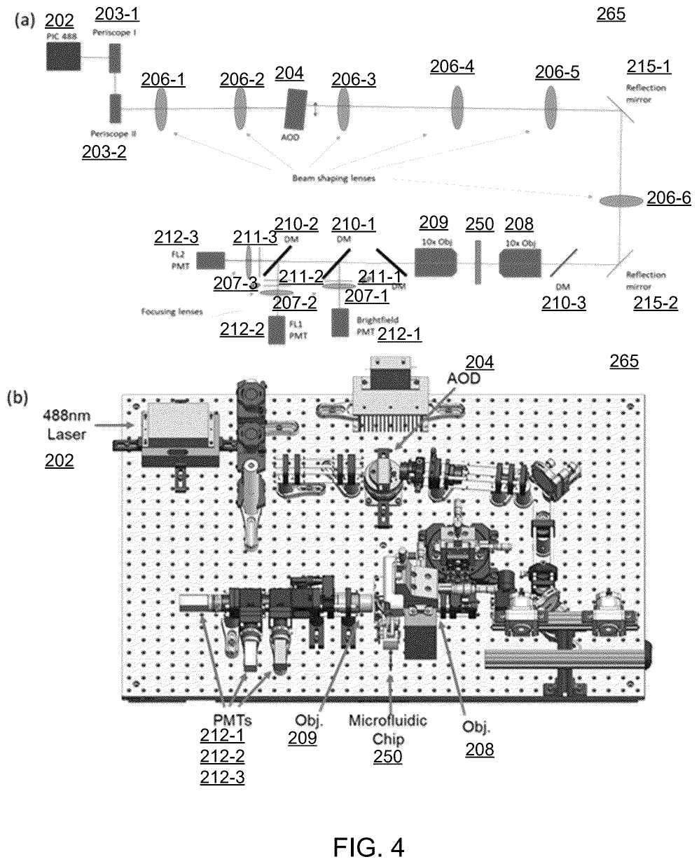

The system 100 implements image-based sorting of the particles in real-time, in which a particle is imaged by the imaging system 120 in the interrogation area and sorted by the actuator 140 in the sorting area 116 in real time and based on a determined property analyzed by the data processing and control unit 130. More descriptions regarding the imaging system 120 may be found elsewhere in the present document. See, e.g., FIGS. 2A and 4, and relevant descriptions thereof. In some embodiments, the system 100 may be user-programmable to sort particles based on one or more of a plurality of image features that are machine-ascertainable from particle images using the gating model in real time. Some example image features include, but are not limited to, intensity, size, shape, or texture of or on individual particles.

FIG. 1B shows a block diagram of an example embodiment of the control command unit 130. In various implementations, the control command unit 130 is embodied on one or more personal computing devices, e.g., including a desktop or laptop computer, one or more computing devices in a computer system or communication network accessible via the Internet (referred to as “the cloud”) including servers and/or databases in the cloud, and/or one or more mobile computing devices, such as a smartphone, tablet, or wearable computer device including a smartwatch or smartglasses. The data processing and control unit 130 includes a processor 131 to process data, and memory 132 in communication with the processor 131 to store and/or buffer data. For example, the processor 131 can include a central processing unit (CPU) or a microcontroller unit (MCU). In some implementations, the processor 131 can include a field-programmable gate-array (FPGA) or a graphics processing unit (GPU). For example, the memory 132 can include and store processor-executable code, which when executed by the processor 131, configures the data processing and control unit 130 to perform various operations, e.g., such as receiving information, commands, and/or data, processing information and data, such as from the imaging system 120, and transmitting or providing processed information/data to another device, such as the actuator 140. To support various functions of the control command unit 130, the memory 132 can store information and data, such as instructions, software, values, images, and other data processed or referenced by the processor 131. For example, various types of Random Access Memory (RAM) devices, Read Only Memory (ROM) devices, Flash Memory devices, and other suitable storage media can be used to implement storage functions of the memory 132. In some implementations, the control command unit 130 includes an input/output (I/O) unit 133 to interface the processor 131 and/or memory 132 to other modules, units or devices. In some embodiments, such as for mobile computing devices, the data processing and control unit 130 includes a wireless communications unit, e.g., such as a transmitter (Tx) or a transmitter/receiver (Tx/Rx) unit. For example, in such embodiments, the I/O unit 133 can interface the processor 131 and memory 132 with the wireless communications unit, e.g., to utilize various types of wireless interfaces compatible with typical data communication standards, which can be used in communications of the control command unit 130 with other devices, e.g., such as between the one or more computers in the cloud and the user device. The data communication standards include, but are not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, and parallel interfaces. In some implementations, the control command unit 130 can interface with other devices using a wired connection via the I/O unit 133. The data processing and control unit 130 can also interface with other external interfaces, sources of data storage, and/or visual or audio display devices, etc. to retrieve and transfer data and information that can be processed by the processor 131, stored in the memory 132, or exhibited on an output unit of a display device or an external device.

In some embodiments, the data processing unit 125 may be implemented in a manner similar to the control command unit 130. In some embodiments, the data processing unit may be implemented on a processor different from the control command unit 130. For example, the data processing unit 125 may be implemented on an FPGA configured to generate particle images for individual particles based on image data acquired by the imaging system 120, while the control command unit 130 may be implemented on a GPU (e.g., a dedicated GPU) configured to determine particle classes for the individual particles by analyzing the particle images using the gating model, and/or control commands for the individual particles based on their respective particle classes. This example configuration may allow parallel processing of the image data and particle classification, thereby improving processing efficiency and reducing latency between the image data acquisition and the sorting decision or actuation, and allowing real time particle sorting while the particles flow through the particle flow device 110.

FIG. 1C shows a diagram of an example process 100A for image-activated particle sorting based on AI gating according to some embodiments of the present document.

Implementations of the process 100A can be performed by the various embodiments of the image-activated particle sorting system including, e.g., systems 100, 200, 300 as illustrated in FIGS. 1A, 2A, and 3A, respectively.

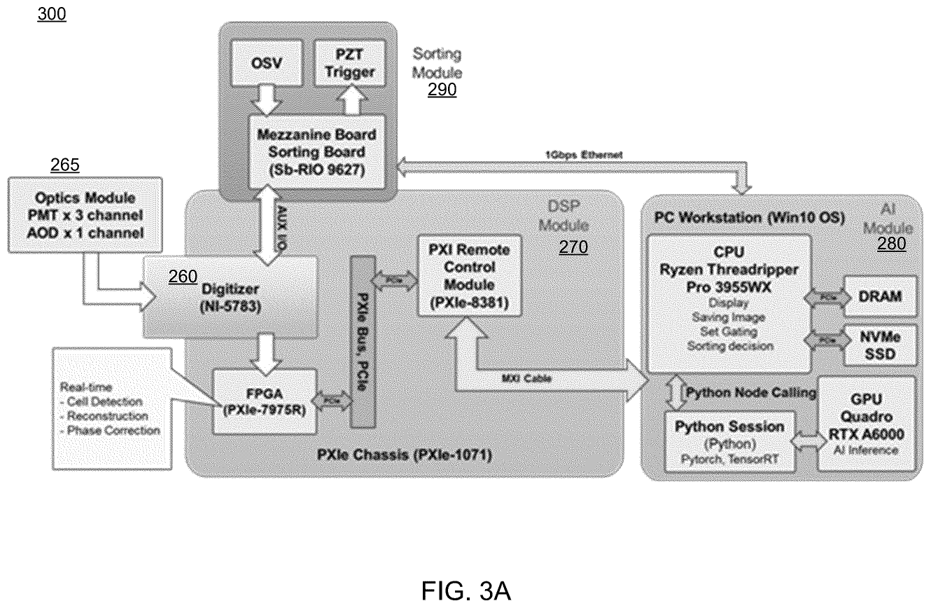

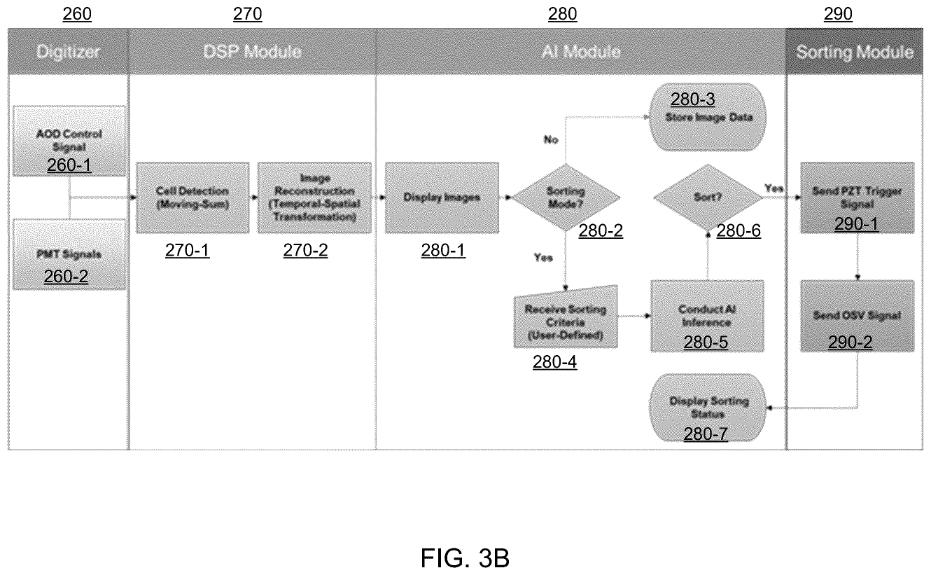

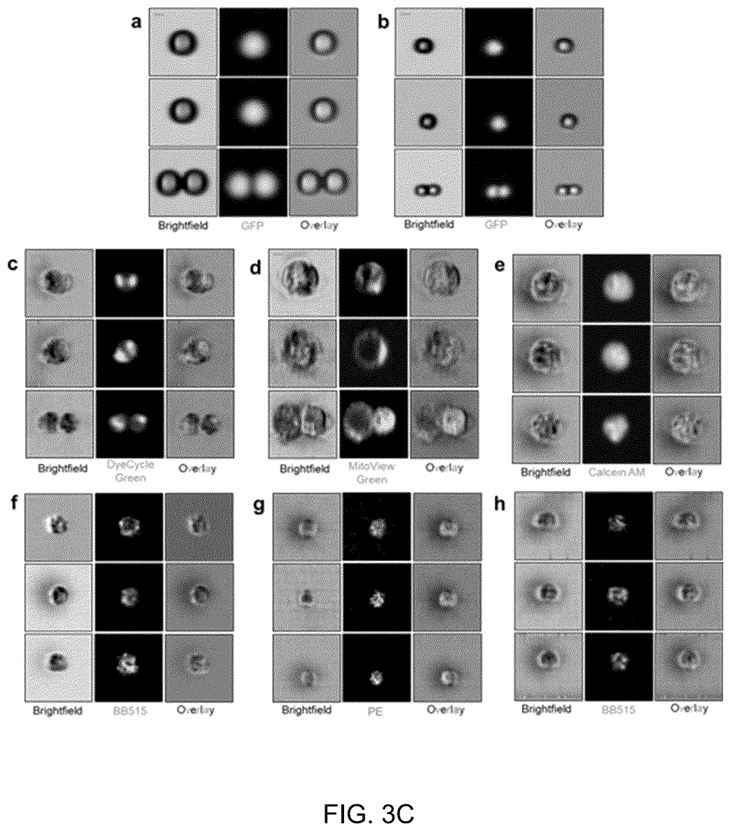

The process 100A may include an operation 155 to obtain, by an imaging system (e.g., imaging system 120) interfaced with a particle flow device (e.g., the particle flow device 110), image data of a particle flowing through a channel (e.g., the channel 112) of the particle flow device. The particle may be labeled (e.g., fluorescently labeled) or label free. Particles may be hydrodynamically focused to the center of a microfluidic channel by a sheath flow in a microfluidic chip. The imaging system may optically interrogate individual particles in the single-particle core flow stream. In some embodiments, the imaging system may emit laser beams to scan individual particles when they individually traverse the interrogation area 114 of the channel 112 on the particle flow device. The imaging system may adjust at least one of the scanning range or the scanning speed to accommodate samples of different particle sizes for a suitable image field of view. For label-free particles, the image data may include bright field signals of the particles. For fluorescently labeled particles, the image data may include fluorescent signals. The signals detected by, e.g., PMTs and the temporal signals are reconstructed to form particle images via real-time processing by, e.g., a data processing unit (e.g., the data processing unit 125, a digitizer 260). The particle images may be two-dimensional images or three-dimensional images. More descriptions regarding the acquisition of the image data and the generation of particle images may be found elsewhere in the present disclosure. See, e.g., the description regarding the digitizer 260 and the digital signal processing (DSP) module 270 in FIGS. 3A and 3B.

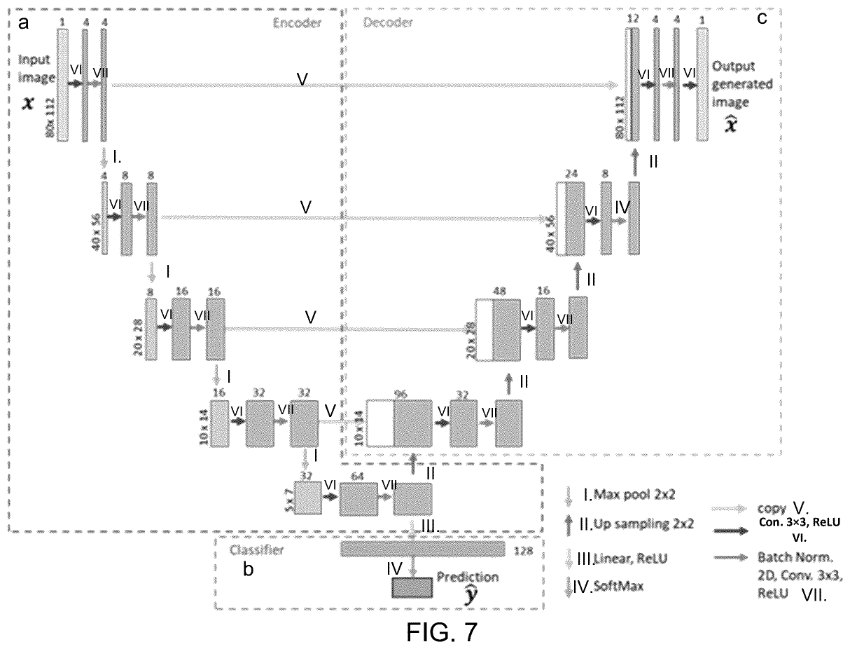

The process 100A includes an operation 165 to produce, by a control command unit (e.g., the control command unit 140), a control command indicative of a particle class of the particle determined based on a gating model and the image data of the particle during the particle flowing in the channel. The gating model may be a convolutional neural network (CNN) trained to conduct real-time AI inference regarding the particle class. The process 100A may include making a sorting decision or a control command based on the particle class. More descriptions regarding the gating model may be found elsewhere in the present document. See, e.g., FIGS. 7-13 and relevant descriptions thereof. More descriptions regarding the determination of the particle class and control command may be found elsewhere in the present disclosure. See, e.g., the description regarding the AI module 280 in FIGS. 3A and 3B.

The process 100A may include an operation to direct the particle into one of a plurality of output paths (e.g., output channels 118) of the particle flow device based on the control command. More descriptions regarding the particle direction may be found elsewhere in the present document. See, e.g., the actuator 140, the sorting module 290 illustrated in FIGS. 1A, 2A, 3A, 3B, and relevant descriptions thereof. The latency between a first time of image capture of a particle by the imaging system to a second time of the particle being directed by the actuator is within a time frame of 15 milliseconds or less. For example, the latency may be less than 10 milliseconds, 8 milliseconds, 6 milliseconds, 5 milliseconds, 3 milliseconds.

FIGS. 2A-2C show diagrams of an image-activated particle sorting microfluidic system 200 in accordance with some embodiments of the image-activated particle sorting system 100. In this exemplary system 200, a scanning laser beam 202 (e.g., 488 nanometer laser) and individual particles (e.g., cells) travel produce the equivalence of a 2D raster scanning system. The bright field and fluorescent signals of the particles are detected by PMTs 212-1 through 212-3 and the temporal signals are reconstructed to form particle images via real-time processing by a digital signal processing (DSP) module 270 (e.g., using an FPGA of the DSP module 270). Meanwhile, each particle image is fed to a convolutional neural network (CNN) to conduct real-time AI inference at an AI module 280. According to the CNN prediction output, the on-chip PZT actuator of a sorting module 290 is triggered to sort out particles. As illustrated, AOD 204 is an acousto-optic deflector; DM 210-1 and 210-2 are dichroic mirrors; IO 208 is a 10×/0.28 NA illumination objective lens; DO 209 is a 10×/0.28 NA detection objective lens; PMTs 212-1 through 212-3 are photomultiplier tubes. For example, a system may employ an acousto-optical deflector (AOD) model OAD948 from Isomet, which operates in coordination with a 25 mW, 488 nm scanning laser sourced from PIC (W488-25FS-025). The scanning laser, which may be adjustable, may probe each cell in the single-cell core flow stream individually. The size of the beam may be modulated by a series of strategically placed beam shaping lenses, ensuring the beam accurately targets each cell.

Further precision in the optical interrogation process may be achieved by employing two 10× objective lenses (0.28 NA Plan Apo, Mitutoyo) situated on opposite sides of the microfluidic chip 250. The illumination laser may form a Gaussian beam with a focal depth of about 25 micrometers, and a full-width-half-maximum (FWHM) circular spot size of 1.6 micrometers at the object plane. The cells may be examined at this plane.

The system may make use of a number of dichroic mirrors (e.g., 210-1, 210-2), focusing lenses (e.g., 207-1 through 207-3) and band-pass optical filters (e.g., 211-1 through 211-3 as illustrated in FIG. 4) to segregate the transmitted laser light and laser-excited fluorescent light into separate photomultiplier tube (PMT) detection channels (e.g., 212-1 through 212-3). These components may enhance the resolution and quality of the images produced, thus increasing the accuracy of the analysis.

The laser scanning range and speed may be adjustable parameters in the system, allowing for the accommodation of samples with varying cell sizes. The maximum field of view that the system 200 may offer is 60×60 micrometers, and it may reach a maximum laser scanning speed of 350 kHz. This adjustability may provide considerable flexibility in the types of cells and samples the system 200 can process. More descriptions regarding the optic portion of the IACS system (system 100, 200, 300) may be found elsewhere in the present disclosure. See, e.g., FIGS. 4-6 and relevant description thereof.

The system 200 may further include a digitizer 260, a DSP module 270, an AI module 280, and a sorting module 290. The digitizer 260 may be configured to capture imaging data of individual particles as illustrated in panel (I) of FIG. 2A. In some embodiments, bright field and fluorescent signals of the particles are detected by PMTs 212-1 through 212-3 and the temporal signals generated by the digitizer 260 are reconstructed to form particle images via real-time processing by the DSP module 270 as in panel (II) of FIG. 2A. An image stack including multiple images of a particle (e.g., a transmission image generated based on the bright field signal, one or more fluorescent images of the particle) may be processed (e.g., overlay, registration) to generate a particle image to be input to the AI module 280 as illustrated in panel (III) of FIG. 2A. The AI module 280 may determine a particle class and/or a sorting decision using a gating model. An actuation may be triggered based on the sorting decision or a corresponding control command for particle direction as illustrated in panel (IV) of FIG. 2A. More descriptions regarding the digitizer 260, a DSP module 270, an AI module 280, and a sorting module 290 may be found elsewhere in the present disclosure. See, e.g., FIGS. 3A and 3B, and relevant description thereof.

FIGS. 2B and 2C show the microfluid chip 250. The microfluid chip 250 may include sheath channels 222-1 and 222-2 configured to facilitate the creation of a sheath flow for particles flowing in the channel 112. Fluidly suspended single particles are hydrodynamically focused in the microfluidic channel by sheath flow, ensuring that the particles travel in the center of the fluidic channel at a (substantially) uniform velocity. The actuator 140 (e.g., a piezoelectric actuator) may be operably coupled to the channel 112 at a sorting junction 115.

The actuator 140 of the system 200 may be configured in communication with the AI module 280 to gate the particle flowing in the sorting area 116 of the sample channel 112 into two or more outlet channels 118 of the microfluidic chip 250. In some embodiments, for example, the distance between the laser interrogation zone 114 and the sorting area 116 can be in a range of 50 micrometers to 1 mm. In implementations, the actuator 140 receives the control command from the AI module 280 in real time, such that the imaging system 120 (including the optical components and the digitizer 260 as illustrated in, e.g., FIGS. 2A, 3A, and 4), the DSP module 270, and the AI module 280 may operate to capture and process the image of each particle while flowing through the laser interrogation zone 114 so that the actuator 140 receives and executes the control command to gate each particle accordingly. For example, in some implementations, the actuator 140 includes a piezoelectric actuator coupled to the channel 112 at the sorting junction 115 to produce deflection that causes a particle to move in a particle direction in the sorting area 116 that directs the particle along a trajectory to one of the two or more of outlet channels 118.

Other examples of features of a particle flow device and/or an actuator that can be used in example embodiments of the devices, systems, and methods in accordance with the disclosed technology are provided in U.S. Pat. No. 9,134,221 B2 entitled “FLUIDIC FLOW CYTOMETRY DEVICES AND PARTICLE SENSING BASED ON SIGNAL ENCODING,” and U.S. Pat. No. 11,016,017 entitled “IMAGE-BASED CELL SORTING SYSTEMS AND METHODS,” the entire content of each of which is incorporated by reference as part of this disclosure for all purposes. Other examples of features of an optical imaging system that can be used in example embodiments of the devices, systems, and methods in accordance are provided in U.S. Pat. No. 10,267,736 entitled “IMAGING FLOW CYTOMETRY USING SPATIAL-TEMPORAL TRANSFORMATION,” the entire content of which is incorporated by reference as part of this disclosure for all purposes.

FIG. 3A shows an exemplary system architecture in accordance with embodiments of the present document. FIG. 3B shows an example real-time data processing pipeline in accordance with embodiments of the present document. The example system 300 may achieve low data processing latency with Artificial Intelligence (AI) inference. For example, this may be accomplished using a hybrid design incorporating a Field-Programmable Gate Array (FPGA), a Central Processing Unit (CPU), and a Graphics Processing Unit (GPU). The system 300 may be programmed in LabVIEW and may be designed to accommodate real-time data processing requirements.

For example, the system 300 may include an optics module 265. More descriptions regarding the optics module 265 may be found elsewhere in the present document. See, e.g., FIGS. 2A and 4-6, and relevant description thereof. The system 300 may include a digitizer 260 (e.g., NI-5783), which samples and converts voltage waveforms (e.g., PMT signals 260-2) at a rate of 25 Mega Samples per Second (MSps). The digitizer 260 may be designed to stream these waveforms continuously to an FPGA (e.g., PXIe-7975R) of the DSP module 270. The FPGA may then apply a threshold to a moving sum window of a user-defined size to detect particles, such as cells, at 270-1. This may be followed by the DSP module 270 reconstructing a particle image via a temporal-spatial transformation algorithm at 270-2. Moreover, the FPGA of the DSP module 270 may carry out a phase shift correction for the image data of the particle or the corresponding particle image. This may be done to rectify the electronic delay that occurs between the control signal 260-1 of the Acousto-Optical Deflector (AOD) and the detected Photo Multiplier Tube (PMT) readout waveforms. This AOD threshold trigger may play a crucial role in initiating the line scan. Once the threshold may be crossed, the system 300 may start gathering output signals until a user-defined image width is obtained. The system 300 may ensure that the output signals collected during the line scan period are sent to a circular buffer while the FPGA waits for a particle to be detected. After a particle is detected at 270-1, the signals may be transferred to a first-in-first-out (FIFO) buffer for image reconstruction at 270-2. These reconstructed images may then be transferred via the PCIe bus to the dynamic random-access memory (DRAM) in the AI module 280.

The AI module 280 may be part of a standalone multi-core PC workstation equipped with a dedicated Nvidia GPU module. In addition, the AI module 280 may provide a Graphical User Interface (GUI) configured to display reconstructed images at 280-1. In some embodiments, the AI module 280 may provide two operating modes for user convenience and a mode selection at 280-2. If a sorting mode is not selected at 280-2, the AI module 280 proceeds with an analysis mode, under which the AI module 280 may save at 280-3 the image data to internal or external solid-state storage disks for offline image processing and AI model training. In contrast, if a sorting mode is not selected at 280-2, the AI module 280 proceeds with the sorting mode. In some embodiments, a user may define sorting criteria (i.e., cell class, confidence level), and the AI module 280 may use a pre-trained gating model to conduct real-time inference to automatically classify the particles, along with a prediction confidence level. Finally, a sorting module 290 may be triggered by the AI module 280 at 290-1 based on the generated sorting decision, which then may trigger an on-chip Piezoelectric Transducer (PZT) actuator to deflect the particle to user-defined downstream channels. An optical sorting verification detector may monitor the sorting outcome at 290 and may send a feedback signal, e.g., an optical sorting verification (OSV) signal, to the AI module 280 to display the sorting yield on the GUI. Merely by way of example, the real-time data processing software for this system may be developed using LabVIEW, featuring a customized Python Node that may call Python code for real-time AI inference in sorting mode.

In some embodiments, the system 300 may operate by sampling temporal waveforms using the digitizer 260 including an analog-to-digital converter (e.g., NI-5783, National Instruments). These waveforms are subsequently transferred to a field-programmable-gate-array (FPGA, PXIe-7975R, National Instruments) of the DSP module 270 for real-time particle image reconstruction utilizing, e.g., temporal-spatial transformation. Reconstructed particle images are then channeled to a standalone PC workstation, hereafter referred to as the AI module 280, via a wide-band PCIe bus. This dedicated AI module 280, equipped with a GPU (Quadro RTX A6000, Nvidia), executes real-time AI inference using a gating model. The AI module 280 is designed to predict particle classes (e.g., cell types) and may assign each AI inference prediction with a corresponding confidence level. Sorting decisions are taken by comparing the AI inference prediction with user-specified particle classes and the assigned classification confidence level. To account for the process's latency, the system 300 includes a clock mechanism that records the duration of the process. If the cumulative processing time is within a preset value, the sorting action is activated, and the sorting decision is transferred via the PCIe bus to the FPGA. The FPGA then controls the on-chip piezoelectric actuator's function, executing the sorting action. In example implementations, the entire data processing operation, which includes AI model inference and PZT actuation, is concluded in less than 3 milliseconds for 99% of the cells in samples, achieving a swift and efficient cell sorting.

In some embodiments, the IACS system described herein may include an imaging apparatus capable of generating both transmission and fluorescent images of particles moving through a microfluidic channel at an approximate velocity of 20 cm/s. FIG. 3C illustrates example beads and cell images captured by an example low-latency IACS system in accordance with embodiments of the present document.

Panel a of FIG. 3C shows results for 15-micrometer fluorescent beads; panel b of FIG. 3C shows results for 7-micrometer fluorescent beads; panel c of FIG. 3C shows results for CHO-ES cells with DNA staining (Vybrant DyeCycle Green); panel d of FIG. 3C shows results for MCF7 cells with mitochondrial staining (Mito View Green); panel e of FIG. 3C shows results for Human iPSC with vitality dye staining (Calcein AM); panel f of FIG. 3C shows results for Human granulocytes with anti-CD66b BB515 immunostaining; panel g of FIG. 3C shows results for human lymphocytes with anti-CD3 and anti-CD19 PE immunostaining; panel h of FIG. 3C shows results for human monocytes with anti-CD14 BB515 immunostaining. Scale bar is 5 micrometers.

The efficacy and versatility of this system disclosed herein have been evidenced in various applications. For instance, it can accurately capture images of both single and multiple fluorescent beads measuring 7 micrometers and 15 micrometers (see FIG. 3, panels a and b). Furthermore, it has demonstrated the capability to resolve the distribution of intracellular DNA and mitochondrial localization in fluorescently labeled CHO-ES and MCF7 cells (see FIG. 3, panels c and d). Notably, the system may be employed to generate brightfield images of human induced pluripotent stem cells (iPSCs), revealing intricate intracellular structures (see FIG. 3, panel e). Moreover, during experiment implementations involving white blood cells, the system has successfully captured the distribution of surface antibodies on immunostained cells (see FIG. 3, panels f-h). As illustrated, this system disclosed herein offers adaptability to capture particles of varying sizes, ranging from 1 to 40 micrometers, and can distinctly display images of doublets.

FIG. 4 illustrates optical components of an example low-latency IACS system in accordance with embodiments of the present document. Panel (a) of FIG. 4 provides the optical schematics and panel (b) provides a computer-aided design (CAD) layout of the optics module 265. All the image acquisition and sorting experiments demonstrated herein may be conducted at a laser scanning frequency of 200 kHz and an image field of view of 35×35 micrometers. These parameters may provide an optimal balance of speed and detail in the example analysis. In an example, optical calibration experiments were conducted to measure the illumination spot size and depth of focus with a complementary metal-oxide-semiconductor (CMOS) camera, model DCC1645C from Thorlabs. These experiments may be performed to establish that the system is calibrated accurately, increasing the precision and reliability of the cell analysis process.

In some embodiments, the optics module 265 may including one or more filters. An example of two-dimensional spatially-varying spatial filter is provided in U.S. Pat. No. 9,074,978 B2 entitled “OPTICAL SPACE-TIME CODING TECHNIQUE IN MICROFLUIDIC DEVICES”, the entire content of which is incorporated by reference as part of this disclosure for all purposes. Additional descriptions of filters suitable to be used in the IACS system disclosed herein may be found in, e.g., U.S. Pat. No. 11,016,017 entitled “IMAGE-BASED CELL SORTING SYSTEMS AND METHODS,” the entire content of each of which is incorporated by reference as part of this disclosure for all purposes.

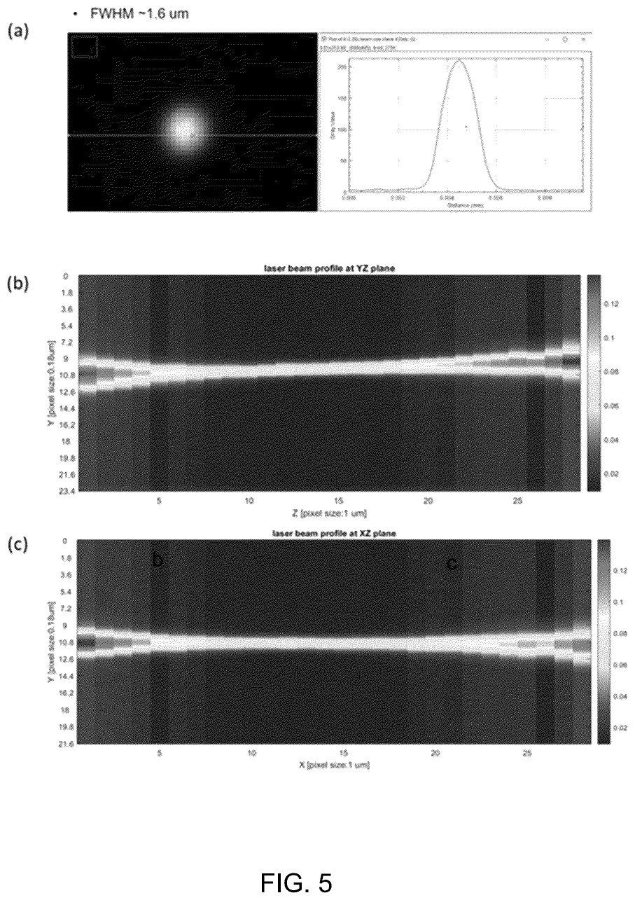

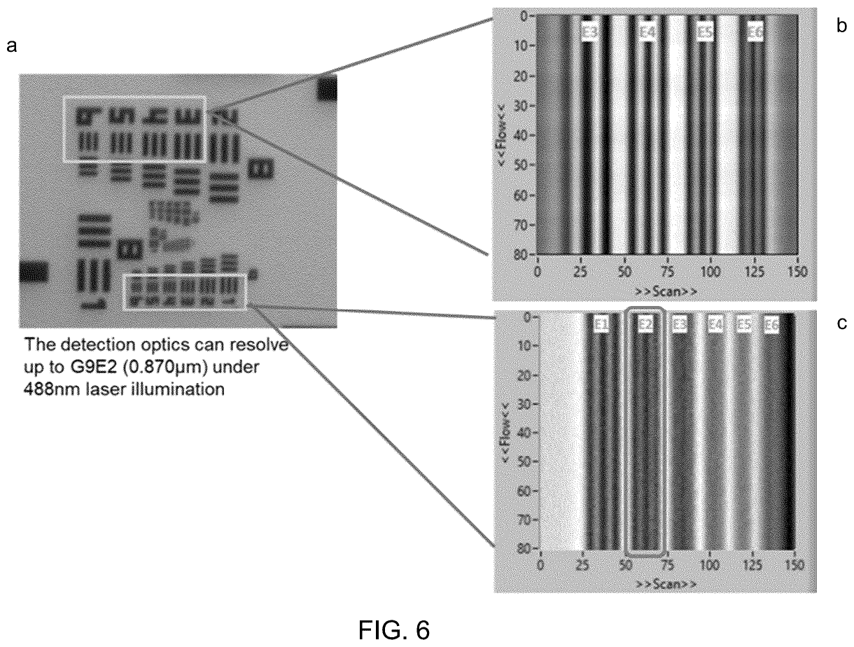

The system's detection optics resolution limit was measured using a high-resolution optical test target, model HIGHRES-1 from Newport. The measured spot size, illumination beam depth of focus, and detection optics resolution limit are presented in FIGS. 5 and 6. Panel (a) of FIG. 5 illustrates illumination spot size measurement, panel (b) of FIG. 5 illustrates measured illumination light depth of focus measurement at a YZ plane, and panel (c) of FIG. 5 illustrates measured illumination light depth of focus measurement at a XZ plane, where X is the laser scanning direction, Y is the cell traveling direction, and Z is the laser propagation direction. Additionally, as illustrated in FIG. 6, it was observed that the detection optics was diffraction-limited by the objective lens numerical aperture (NA) and excitation laser light wavelength. This may provide a limit on the system's resolution but also confirms that the system may be operating at the limit of what is physically possible, improving or maximizing the level of detail the system can extract from the examined cells.

The AI based gating model for real-time data processing may be trained using a Convolutional Neural Network (CNN) model training process. For example, the training system for training a CNN model may utilize a custom MATLAB image preprocessing code to extract conventional image features, leading to the generation of human interpretable image features and a preprocessed image dataset. FCS Express software may be employed to import the list of these extracted features, enabling the user to define gating to select targeted image data for the CNN model training. The selected image indices may be exported by FCS Express, which are then prepared for CNN model training via a MATLAB code. Table 1 lists the image features extracted by the MATLAB program. The average processing time for a dataset comprising 20,000 images may be between 5 to 10 minutes with this exemplary approach.

TABLE 1
Human-vision image features extracted in image preprocessing step

| Feature Category | Image feature Name |
|---|---|
| Intensity | Raw Integral Intensity, Raw Maximum Intensity, Raw |
| | Average Intensity, Masked Integral Intensity, Masked |
| | Maximum Intensity, Masked Average Intensity |
| Size | Major Axis Length, Minor Axis Length, Area |
| Shape | Aspect Ratio |
| Texture | Root-Mean-Square Gradient Magnitude |

To expedite the CNN model training and achieve low-latency model inference for sorting, a customized 2D UNet may be developed. FIG. 7 illustrates an exemplary architecture of the 2D UNet in accordance with embodiments of the present document. This UNet may be trained using few labeled images and maintains a reasonable training time. Autoencoder utilization and conducting latent space may bolster classification performance. The developed UNet model may have superior performance compared with the ResNet-18 CNN architecture, possessing a faster convergence rate and fewer model parameters than other CNN architectures like VGG or InceptionNet.

In this example as illustrated in FIG. 7, the number of channels (or referred to as channel count) is denoted on top of each box. The feature map size is shown at the lower-left edge of each box. The arrows marked by different Roman numerals denote different operations. The 2D UNet may include a contracting path to encode image features and input images, and an upsampling path that may work in unison with the higher resolution features passing the convolution layers to generate an output image of the same dimension as the input image. The architecture may also include a fully connected layer and Softmax layer connected to the latent space for classification decisions. The upsampling path may receive features from a latent space and may combine these with higher resolution features, which may have passed through the convolution layers. The path's main function may be to generate an output image that may be dimensionally identical to the input image. This feature may ensure that the extracted features may preserve the original spatial configuration of the image, providing an advantage in certain imaging tasks. An integral part of this system may be its two-part dynamic: the contracting path, which may serve as an encoder, and the upsampling path, which may function as a decoder. The contracting path may capture and condense complex image features into a latent space representation. The upsampling path may decode the condensed representation, recreating the high resolution features that may then be used to generate the output image. The output of the Softmax layer can be written as:

$${\hat{y}}_{i} = \frac{{e}^{{x}_{i}}}{{\sum }_{j = 1}^{C} {e}^{{x}_{j}}} , i = 1 , 2 , ... , C \qquad ( 1 )$$

where x is the input vectors, C is the number of particle classes.

Additionally, the system may comprise a fully connected layer and a Softmax layer that may collaboratively function as a classifier. The fully connected layer may capture high-level features from the output of the upsampling path, condensing them into a feature vector. The Softmax layer may then process this vector, producing probabilities for each class in the classification task. This combination may ensure accurate and probabilistically nuanced class assignments for the input images. In the model training process, a weighted loss may be used, incorporating mini-batch averaged cross-entropy loss and mean-square error loss between input and generated output image pixel values. The total loss may be balanced through a weight coefficient. This process may provide an effective method to manage classification error. The averaged cross-entropy loss L_{CE} can be expressed as

$${L}_{CE} = - \frac{1}{N} {\sum }_{i = 1}^{N} {y}_{i} ( x ) \cdot log ( {\hat{y}}_{i} ( x ) ) \qquad ( 2 )$$

where yᵢ is the ground truth class vector, ŷᵢ is the predicted class vector, and N is the data size in the mini-batch. The mini-batch averaged mean-square error loss L_{MSE} can be expressed as

$${L}_{MSE} ( x , \hat{x} ) = \frac{1}{N} {\sum }_{j = 1}^{N} {\sum }_{i = 1}^{M} {( {x}_{i , j} - {\hat{x}}_{i , j} )}^{2} \qquad ( 3 )$$

where x and x are the input image and generated image vectors, respectively, M is the flattened image vector dimension, and N is the data size in the mini-batch.

The weighted total loss L is defined as

$$L = w \cdot {L}_{CE} + ( 1 - w ) \cdot {L}_{MSE} \qquad ( 4 )$$

where w is the weight coefficient to balance the loss function.

The UNet model architecture may be optimized by conducting a CNN model architecture search, aiming to reduce the initial convolutional kernel number in the UNet model. As part of the model optimization, a stratified 5-fold cross-validation (CV) approach may be employed to train and assess the performance of the UNet models. In this process, for each fold, the training data may be augmented by conducting random horizontal and vertical flips on the image data. Following the augmentation, the model may then be validated using instances from the validation set. The performance of the model may be evaluated by calculating the balanced classification accuracy, an accuracy metric that may not favor classifiers that exploit class imbalance by biasing toward the majority class. The balanced accuracy σ̅ is the arithmetic mean of class-specific accuracies and is calculated as

$$\overline{σ} = \frac{1}{C} {\sum }_{i = 1}^{C} {σ}_{i} \qquad ( 5 )$$

where σᵢ is the class-specific accuracy, and C is the number of particle classes. In an example training, a dataset including 15,000 images obtained from a white blood cell imaging experiment were employed to carry out this model architecture search. To maintain a balanced data occurrence, 5,000 cell images were used for each cell type. Additionally, to examine the impact on inference time utilizing different GPU acceleration frameworks, a comparative analysis between Pytorch and TensorRT frameworks were performed during the UNet architecture search. In some embodiments, deep learning model training and performance tests may be conducted on the same computer system or different computing systems, situated within the AI module of the low-latency IACS system. The deep learning development may be performed under specific frameworks including, e.g., Python 3.6.8, Pytorch 1.10.2, and TensorRT 8.2.2.1. The use of these frameworks may contribute to the efficiency and functionality of the model training and optimization processes, offering robustness and reliability to the overall system.

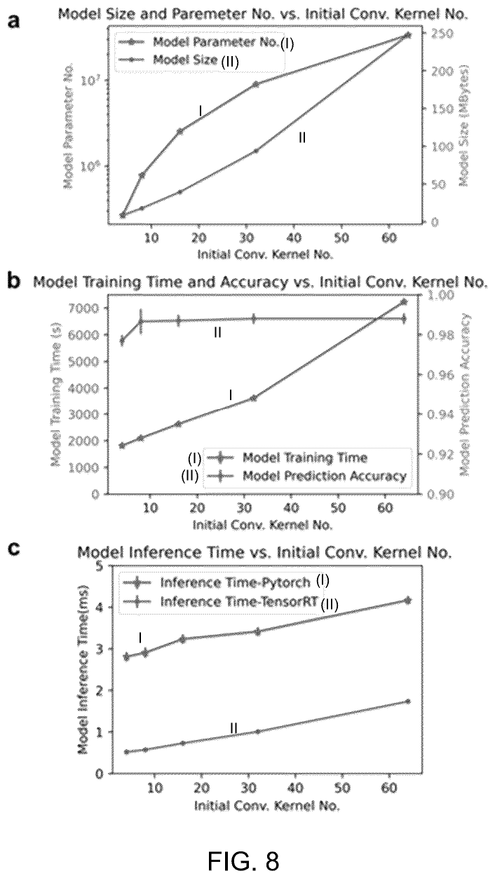

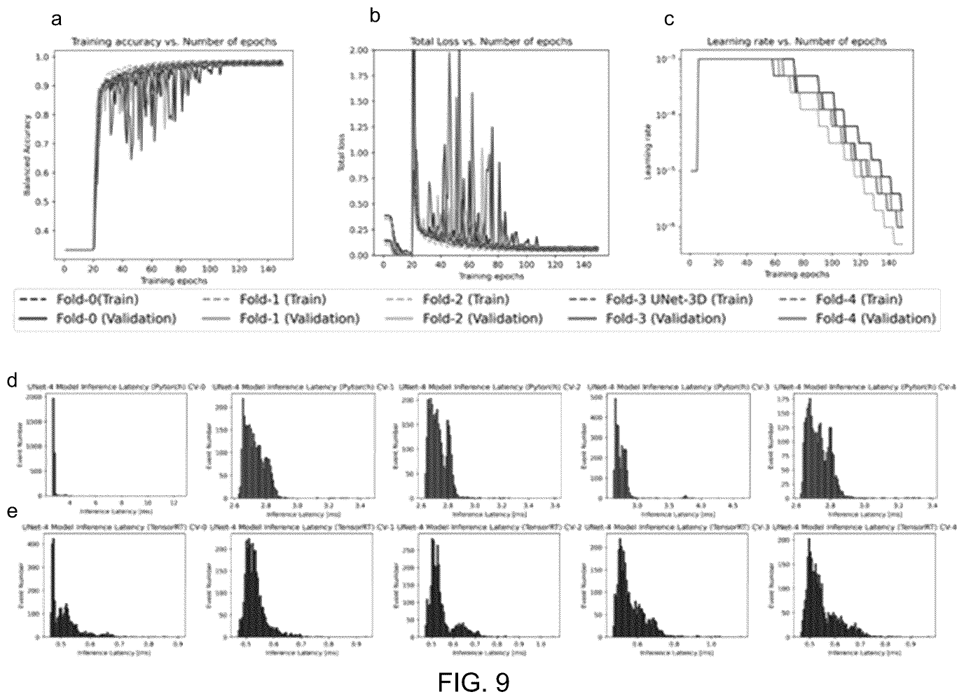

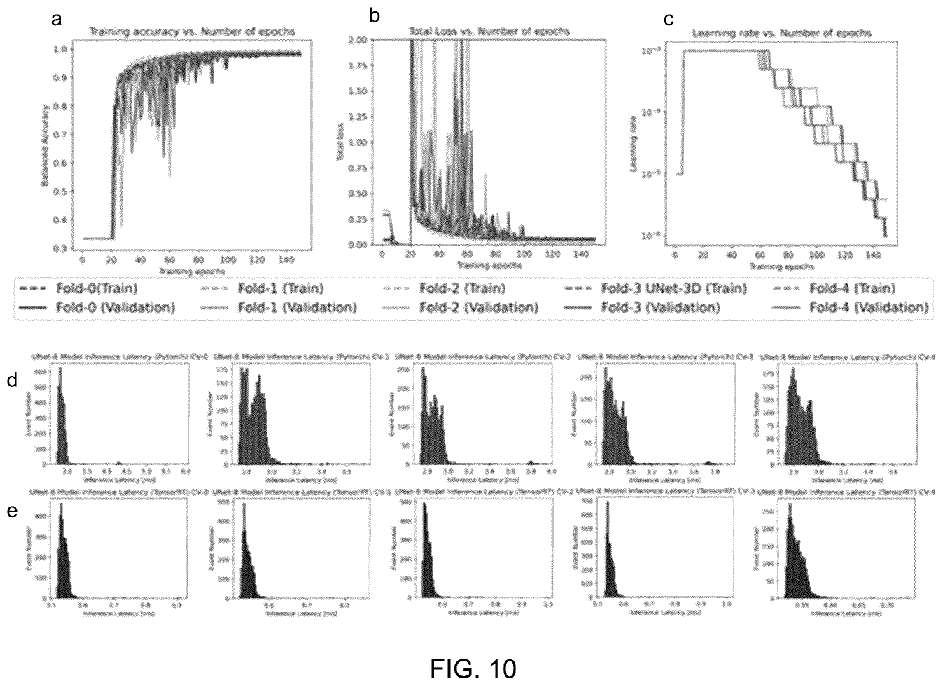

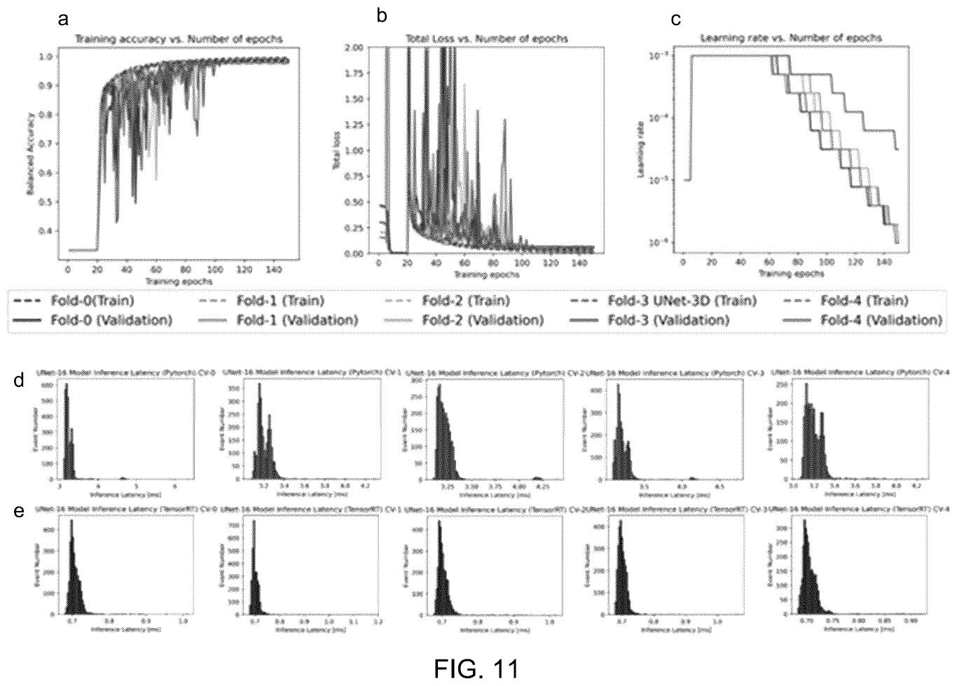

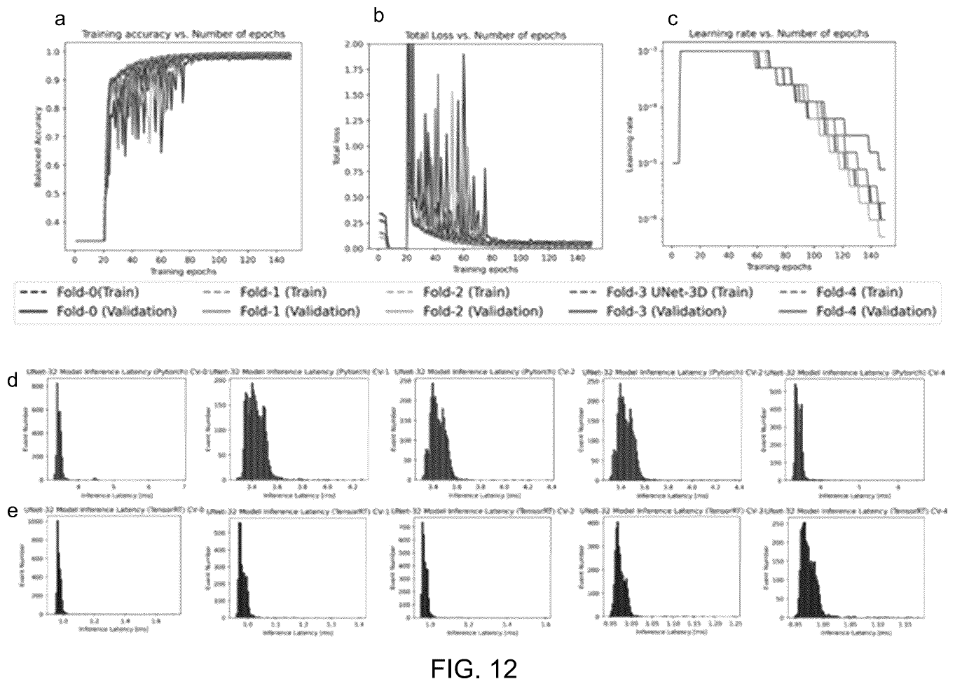

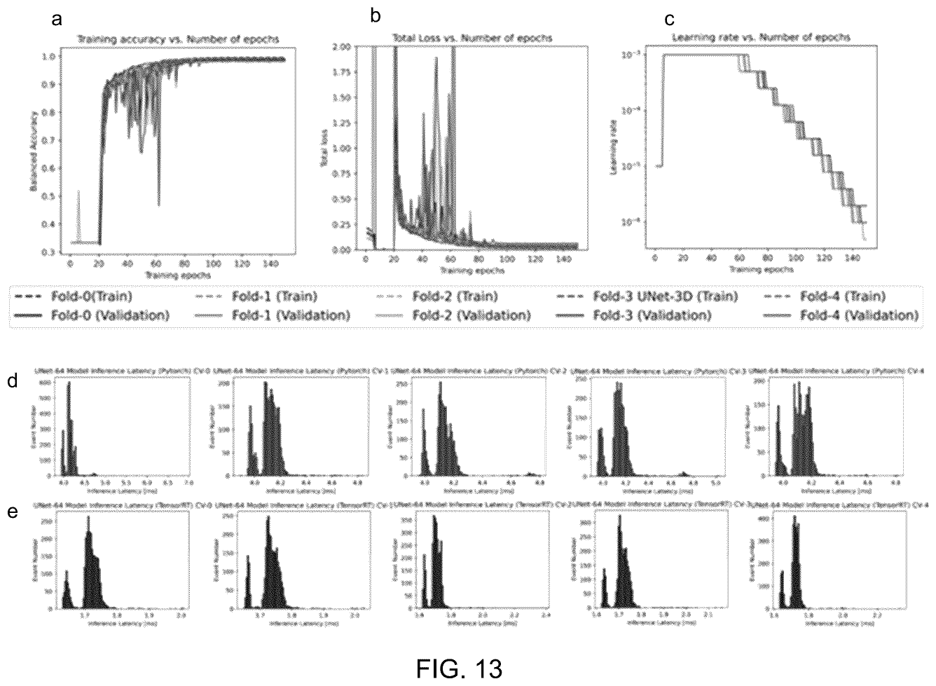

In example implementations, through an extensive CNN model architecture search, a UNet CNN model may be optimized for 2-part or 3-part particle classification. The results indicate that the initial convolutional kernel number (or kernel count) may significantly affect the model size, parameter number, training time, and inference time, while having a relatively low impact on the model prediction accuracy within our system. Merely by way of example, by reducing the initial convolutional kernel count from 64 to 4, the model parameter and model size by approximately may be reduced by 100-fold as illustrated in FIG. 8. Panels a, b, and c of FIG. 8 illustrate custom UNet model optimization on model size, training time, and inference time. Specifically, panel a of FIG. 8 shows model size and parameter number vs. Initial convolutional kernel number; panel b of FIG. 8 shows model training time vs. Initial convolutional kernel number; panel c of FIG. 8 shows model inference time under Pytorch and TensorRT frameworks vs. Initial convolutional kernel number; error bars±SD. This reduction may not only enhance the efficiency of the system but also decrease the total training time and model inference time. In this example, a 4-fold reduction was achieved in model training time from 7200 seconds to less than 1800 seconds during a CNN training process using a dataset of 15,000 images, while maintaining a high model prediction accuracy of 0.977. Moreover, with leveraging the optimized GPU acceleration framework under the TensorRT framework, an approximate 5-fold reduction in the model inference time was achieved compared to the Pytorch framework. The shrinking of the initial convolutional kernel number from 64 to 4 further improved the model inference time from 1.735 milliseconds to 0.518 milliseconds under the TensorRT framework (panel c of FIG. 8). This significant improvement may contribute to reducing the sorting latency for real-time CNN inference during the sorting experiment. Further details about CNN model training and inference time optimization can be found elsewhere in the present document. See, e.g., FIGS. 9-13, which provide comprehensive insights into the specific methodologies employed to achieve the improved efficiency and performance of the system. FIG. 9 displays the UNet training curves and the UNet inference time when the initial convolutional kernel size is equal to 4. FIG. 10 shows the UNet training curves and the UNet inference time when the initial convolutional kernel size is 8. FIG. 11 reveals the UNet training curves and the UNet inference time when the initial convolutional kernel size is set to 16. FIG. 12 depicts the UNet training curves and the UNet inference time when the initial convolutional kernel size is 32. FIG. 13 demonstrates the UNet training curves and the UNet inference time when the initial convolutional kernel size is 64.

The described system represents a novel approach for optimizing a CNN model architecture, e.g., the UNet CNN model, to conduct efficient particle sorting based on imaging data of individual particles. By reducing the initial convolutional kernel number, the system achieves substantial improvements in model parameter reduction, model size reduction, training time reduction, and inference time reduction. These advancements result in enhanced system efficiency and real-time performance, while maintaining high model prediction accuracy. The optimizations made using the TensorRT framework further contribute to the reduction of model inference time, ensuring low sorting latency for real-time CNN inference. The detailed information provided in this description, along with the supplementary figures, demonstrates the novelty and effectiveness of the system in particle classification applications.

Two types of sorting experiments, beads sorting experiments and WBC sorting experiments were conducted to demonstrate the disclosed system's significant capabilities. The beads sorting experiments showcase the sorting of beads of targeted size from a mixture of 7-micrometer and 15-micrometer polystyrene (PS) fluorescent microsphere beads. To illustrate the system's competence in cell sorting, a second experiment, sorting 3-part human white blood cells (WBCs), were performed to segregate the targeted WBC type from leukocyte samples.

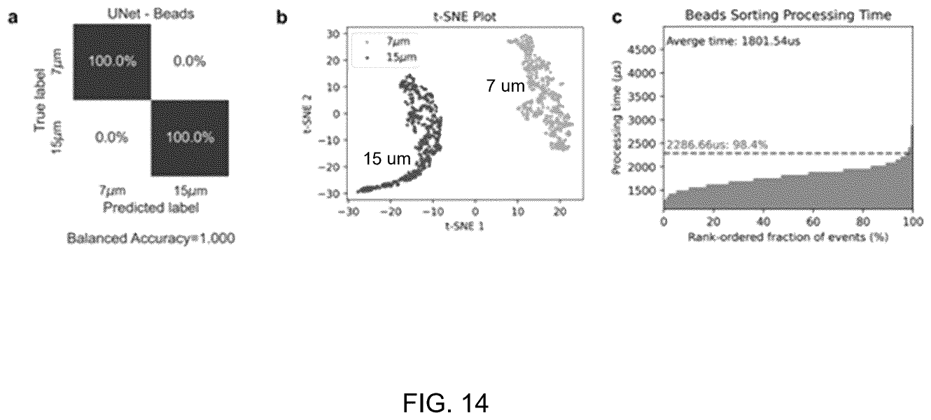

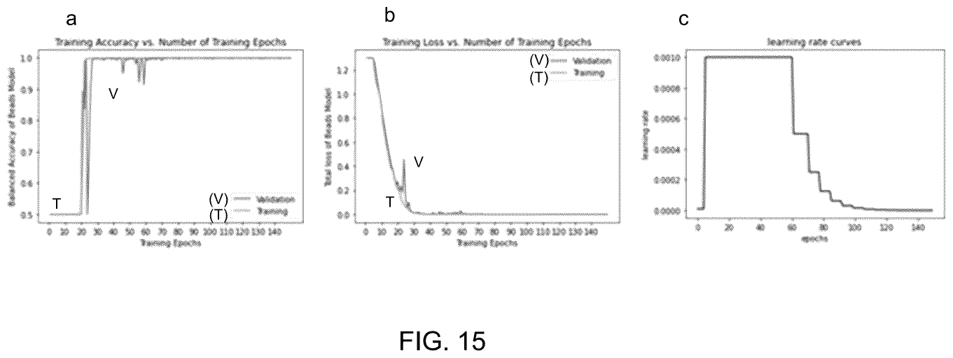

In the beads sorting experiment, separate 7-micrometer and 15-micrometer PS beads samples were prepared and processed through the system in an analysis mode to accumulate a training dataset. This dataset included a total of 4,000 images, evenly divided between the two bead sizes. A two-part image classification training was conducted to train the UNet model, utilizing an 80/20 train/validation split of the dataset. Performance evaluation of the pre-trained model was carried out via generation of a confusion matrix of the classification results from the validation dataset. Upon achieving a prediction confidence level of over 99% from the AI based gating model, the pre-trained CNN model was deployed for bead sorting. Sorted 15-micrometer PS beads from the mixture were analyzed using a commercial flow cytometer (Accuri C6 plus, BD Biosciences), and the outputs from the sorting and waste channels were examined under a fluorescence microscope after enrichment via centrifugation. In the bead sorting experiment, the training progress and classification performance of the pre-trained CNN model were evaluate. The model training process completed within 540 seconds using a dataset of 4000 images for 2-part classification. The pre-trained CNN model achieved a balanced prediction accuracy of 100%. The t-SNE visualization demonstrates distinct separation of the 7-micrometer and 15-micrometer bead clusters (panels a and b of FIG. 14). In panel a of FIG. 14, the confusion matrix showcases the performance of the bead sorting, illustrating the classification accuracy for different bead sizes. Panel b of FIG. 14 displays the t-SNE visualization, which effectively separates and visualizes the clusters of 7-micrometer and 15-micrometer beads, demonstrating the successful classification. Detailed information about the model training process for bead sorting is in panels a-c of FIG. 15, which illustrate UNet training curves for beads sorting experiment. During the bead sorting experiment, the data processing time of the deployed pre-trained CNN model was monitored. The processing time of 1118 sorting events records, and the rank-ordered processing time distribution is in panel c of FIG. 14, which provides insights into the data processing time during the bead sorting experiment, presenting the rank-ordered distribution of the processing time for the sorting events. As illustrated, 98.4% of the events were processed within 2287 microseconds, with an average data processing time of 1802 microseconds.

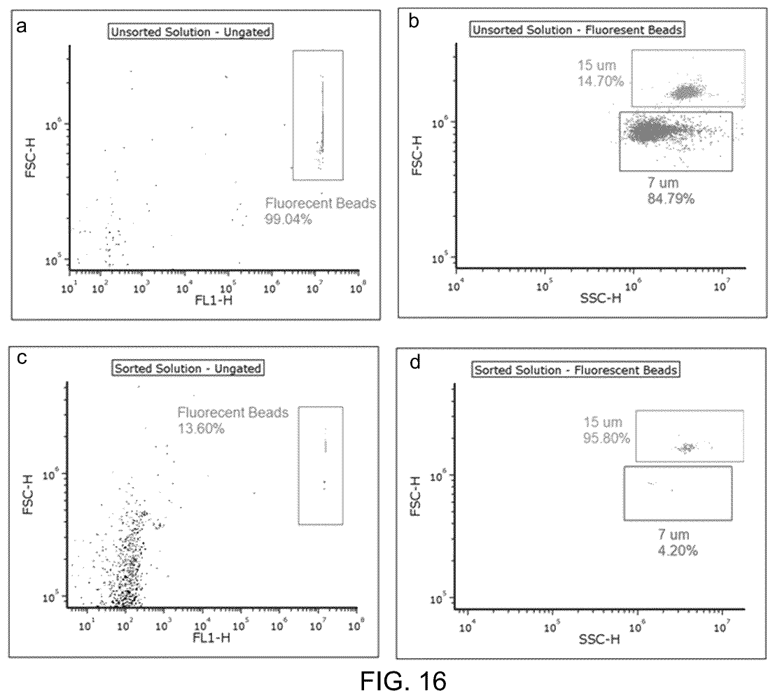

FIG. 16 shows beads sorting experiment pre-sorting and post-sorting Accuri particle composition analysis in accordance with embodiments of the present document. To measure the purity of the AI-inferred bead sorting, particle composition analysis were performed on the collected samples using a commercial cytometer (Accuri C6, BD Biosciences), resulting in a 96.6% purity as illustrated. Pre-sorting data is shown in panels a and b of FIG. 16 and also and Table 2, and post-sorting data is shown in panels c and d of FIG. 16 and also Table 3. Microscopic images of pre-sorting, post-sorting, and waste samples are shown in FIG. 31, which provides consistent results with the confirmatory flow cytometer analysis.

TABLE 2

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0408 7, 15 um | Fluorescent | None | 8118 | n/a | 945784.45 | 100.0 | 99.04 |
| | Beads.c6 | Beads | | | | | | |
| | (Sample | | | | | | | |
| | A06.fcs) | | | | | | | |
| 1 | 0408 7, 15 um | Fluorescent | Fluorescent | 8118 | n/a | 945784.45 | 100.0 | 99.04 |
| | Beads.c6 | Beads | Beads | | | | | |
| | (Sample | | | | | | | |
| | A06.fcs) | | | | | | | |
| 1 | 0408 7, 15 um | Fluorescent | 15 um | 1193 | 3936707.84 | 1658058.34 | 14.70 | 14.55 |
| | Beads.c6 | Beads | | | | | | |
| | (Sample | | | | | | | |
| | A06.fcs) | | | | | | | |
| 1 | 0408 7, 15 um | Fluorescent | 7 um | 6883 | 1624214.15 | 857670.07 | 84.79 | 83.97 |
| | Beads.c6 | Beads | | | | | | |
| | (Sample | | | | | | | |
| | A06.fcs) | | | | | | | |

TABLE 3

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0408 7, 15 um | Fluorescent | None | 119 | 3790660.78 | 1627287.35 | 100.00 | 13.60 |
| | Beads.c6 | Beads | | | | | | |
| | (Sample | | | | | | | |
| | C04.fcs) | | | | | | | |
| 1 | 0408 7, 15 um | Fluorescent | Fluorescent | 119 | 3790660.78 | 1627287.35 | 100.00 | 13.60 |
| | Beads.c6 | Beads | Beads | | | | | |
| | (Sample | | | | | | | |
| | C04.fcs) | | | | | | | |
| 1 | 0408 7, 15 um | Fluorescent | 15 um | 114 | 3929832.78 | 1675965.80 | 95.80 | 13.03 |
| | Beads.c6 | Beads | | | | | | |
| | (Sample | | | | | | | |
| | C04.fcs) | | | | | | | |
| 1 | 0408 7, 15 um | Fluorescent | 7 um | 5 | 1666043.38 | 831004.97 | 4.20 | 0.57 |
| | Beads.c6 | Beads | | | | | | |
| | (Sample | | | | | | | |
| | C04.fcs) | | | | | | | |

In the WBC sorting experiments, WBC samples were immunostained with an antibody panel to provide ground truth labels for each cell type. The immunostained WBC samples were processed to derive the training dataset, encompassing a total of 17,876 cell images. The UNet model was trained via a three-part image classification process using an 80/20 train/validation split of the stratified dataset. With an AI model prediction confidence level exceeding 99%, the pre-trained CNN model was deployed for cell sorting. For each sorting experiment, the target cell type was fluorescently labeled with one color and other cell types with another color using the antibodies panel for the sole purpose of post-sorting performance evaluation (Table 4) while the AI inference and the sorting decision were entirely based on the label-free, transmission images.

TABLE 4
Antibody panel design for human white blood cell
training data collection ground truth labeling

| | Ground Truth | Ground Truth | |
|---|---|---|---|
| | Labelling 1 | Labelling 2 | Unlabeled |
| Panel 1 | Monocytes (PE | Lymphocytes (BB515 | Granulocytes |
| | anti-human CD14 | Mouse Anti-Human | and others |
| | Antibody) | CD3 and CD19) | |
| Panel 2 | Granulocytes (PE | Lymphocytes (BB515 | Monocytes |
| | anti-human CD66b | Mouse Anti-Human | and others |
| | Antibody) | CD3 and CD19) | |
| Panel 3 | Lymphocytes (PE Mouse | Granulocytes | Monocytes |
| | Anti-Human CD3 and | (BB515 anti-human | and others |
| | CD19 antibodies) | CD66b Antibody) | |

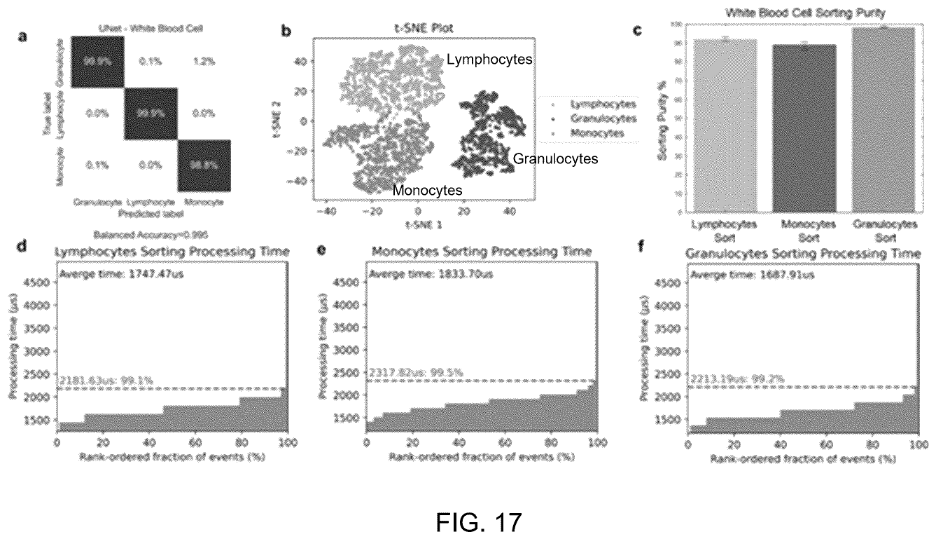

To confirm the system's performance, the sorted and waste samples were analyzed using a commercial flow cytometer (Accuri C6 plus, BD Biosciences), and fluorescent signals of each WBC cell type were utilized to assess the sorting purity. The sorting purity measurement was evaluated by counting the ratio between the sorted target particle number and the total sorted particle number described by Eqn. (6):

$$Sorting Purity = \frac{{N}_{target}}{{N}_{target} + {N}_{non - target}} \qquad ( 6 )$$

where N_{target} is the sorted target particle number (or referred to as particle count) and N_{non-target} is the sorted non-target particle number (or referred to as particle count). During the sorting experiment, the event processing time to evaluate the sorting latency were observed and recorded. In some embodiments, label-free white blood cell (WBC) classification and sorting have significant advantages over biomarker labeling approaches in terms of avoiding cell degradation and minimizing morphological changes. Existing CNN-based label-free WBC classification systems lack real-time AI inferencing capabilities for sorting. However, the customized UNet CNN model as disclosed herein demonstrates high-accuracy and feasible 3-part WBC type classification. The CNN model training completed within 40 minutes using an approximate training set of 18,000 images. The pre-trained CNN model yielded a balanced classification accuracy of 99.5% for 3-part WBC type classification. The t-SNE visualization demonstrates well-separated clusters of the cell groups (FIG. 17).

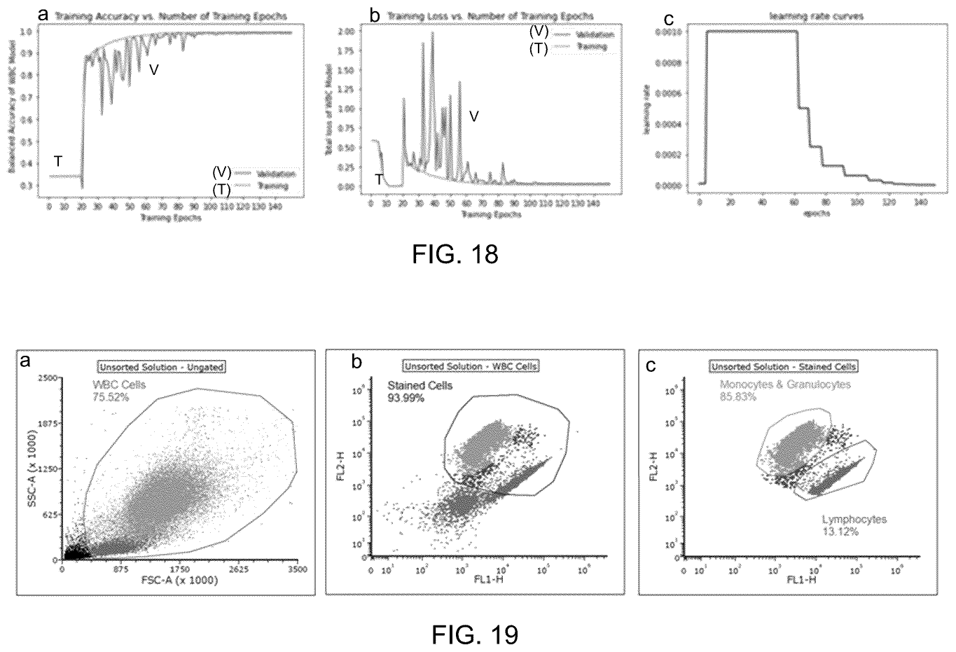

Panel a of FIG. 17 presents a confusion matrix that details the outcomes of the white blood cell sorting experiment. Panel b of FIG. 17 presents a t-SNE visualization that illustrates the classification of the white blood cells. Further details regarding the model training process for WBC sorting are in FIG. 18, which shows the training curves of the UNet model for the human white blood cell sorting experiment, illustrating the training progress and model performance.

In the AI-inferred WBC sorting experiments, a relatively small number of cells were sorted to separate lymphocyte, monocyte, and granulocyte groups. More than 99% of the sorting events processed within 2.312 milliseconds, with average data processing times ranging from 1.687 to 1.834 milliseconds (panels d-f of FIG. 17). Three replicates of sorted samples were collected for sorting purity analysis. The average sorting purities for lymphocytes, monocytes, and granulocytes are 92.0%, 89.05%, and 98.4%, respectively (panel c of FIG. 17). Sorting purity variation related to or was influenced by the initial cell compositions, with lymphocytes, monocytes, and granulocytes including 13.12%, 6.51%, and 79.48% of the initial samples, respectively.

FIGS. 19-30 and Tables 5-16 illustrate additional post-sorting cell composition analysis. FIG. 19 and Table 5 present the Accuri particle composition analysis performed before the lymphocyte sorting experiment, providing information about the composition of the initial lymphocyte sample in accordance with embodiments of the present document.

TABLE 5

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 45235 | 772121.41 | n/a | 100.0 | 100.0 |
| | (Sample | | | | | | | |
| | C08.fcs) | | | | | | | |
| 1 | 0330.c6 | No Gate | WBC Cells | 34161 | 1291342.02 | 565380.62 | 75.52 | 75.52 |
| | (Sample | | | | | | | |
| | C08.fcs) | | | | | | | |
| 1 | 0330.c6 | No Gate | Stained Cells | 32107 | 1334785.89 | 611680.77 | 70.98 | 70.98 |
| | (Sample | | | | | | | |
| | C08.fcs) | | | | | | | |
| 1 | 0330.c6 | No Gate | Lymphocytes | 4211 | 752527.13 | 152309.24 | 9.31 | 9.31 |
| | (Sample | | | | | | | |
| | C08.fcs) | | | | | | | |
| 1 | 0330.c6 | No Gate | Monocytes & | 27559 | 1455565.59 | 755799.75 | 60.92 | 60.92 |
| | (Sample | | Granulocytes | | | | | |
| | C08.fcs) | | | | | | | |

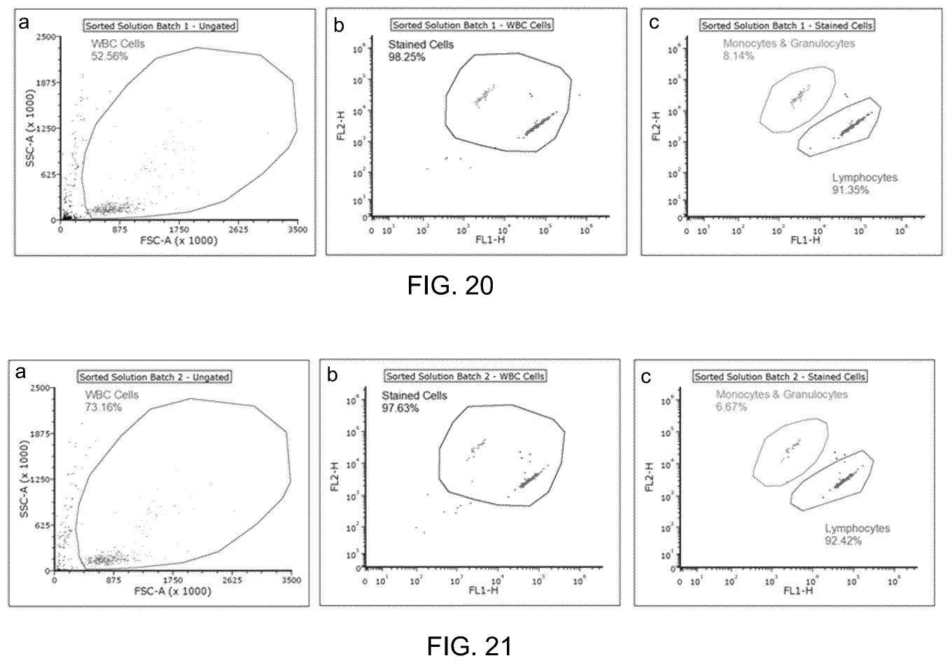

FIG. 20 and Table 6 show the Accuri particle composition analysis for the post-sorting batch 1 solution in the lymphocyte sorting experiment, offering insights into the composition of the sorted lymphocytes in accordance with embodiments of the present document.

TABLE 6

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 761 | 323968.30 | 113379.17 | 100.00 | 100.00 |
| | (Sample | | | | | | | |
| | C01.fcs) | | | | | | | |
| 1 | 0330.c6 | No Gate | WBC Cells | 400 | 827159.32 | 187499.91 | 52.56 | 52.56 |
| | (Sample | | | | | | | |
| | C01.fcs) | | | | | | | |
| 1 | 0330.c6 | No Gate | Stained Cells | 393 | 830107.88 | 185616.49 | 51.64 | 51.64 |
| | (Sample | | | | | | | |
| | C01.fcs) | | | | | | | |
| 1 | 0330.c6 | No Gate | Lymphocytes | 359 | 780452.42 | 161480.41 | 47.17 | 47.17 |
| | (Sample | | | | | | | |
| | C01.fcs) | | | | | | | |
| 1 | 0330.c6 | No Gate | Monocytes & | 32 | 1564559.08 | 812723.93 | 4.20 | 4.20 |
| | (Sample | | Granulocytes | | | | | |
| | C01.fcs) | | | | | | | |

FIG. 21 and Table 7 show the Accuri particle composition analysis for the post-sorting batch 2 solution in the lymphocyte sorting experiment, providing information about the composition of the sorted lymphocytes in accordance with embodiments of the present document.

TABLE 7

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 462 | 519018.98 | 186654.64 | 100.00 | 100.00 |
| | (Sample | | | | | | | |
| | C02.fcs) | | | | | | | |
| 1 | 0330.c6 | No Gate | WBC Cells | 338 | 807156.48 | 177324.89 | 73.16 | 73.16 |
| | (Sample | | | | | | | |
| | C02.fcs) | | | | | | | |
| 1 | 0330.c6 | No Gate | Stained Cells | 330 | 814232.59 | 175531.57 | 71.43 | 71.43 |
| | (Sample | | | | | | | |
| | C02.fcs) | | | | | | | |
| 1 | 0330.c6 | No Gate | Lymphocytes | 305 | 775389.52 | 156307.07 | 66.02 | 66.02 |
| | (Sample | | | | | | | |
| | C02.fcs) | | | | | | | |
| 1 | 0330.c6 | No Gate | Monocytes & | 22 | 1440260.54 | 695658.46 | 4.76 | 4.76 |
| | (Sample | | Granulocytes | | | | | |
| | C02.fcs) | | | | | | | |

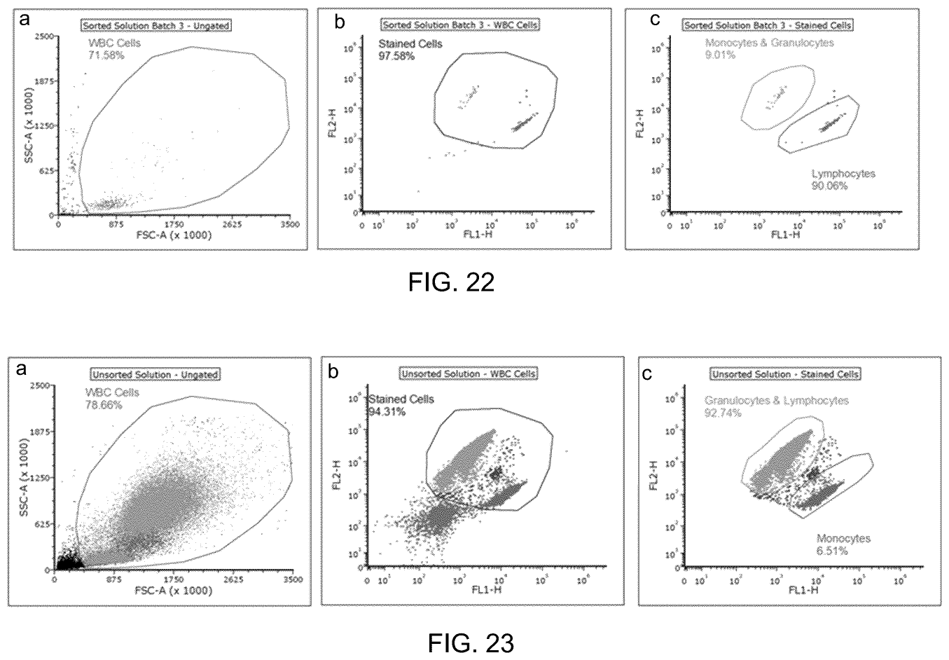

FIG. 22 and Table 8 show the Accuri particle composition analysis for the post-sorting batch 3 solution in the lymphocyte sorting experiment, offering insights into the composition of the sorted lymphocytes in accordance with embodiments of the present document.

TABLE 8

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 461 | 523024.39 | 186470.24 | 100.00 | 100.00 |
| | (Sample | | | | | | | |
| | C03.fcs) | | | | | | | |
| 1 | 0330.c6 | No Gate | WBC Cells | 330 | 831910.86 | 185339.67 | 71.58 | 71.58 |
| | (Sample | | | | | | | |
| | C03.fcs) | | | | | | | |
| 1 | 0330.c6 | No Gate | Stained Cells | 322 | 829859.89 | 182629.47 | 69.85 | 69.85 |
| | (Sample | | | | | | | |
| | C03.fcs) | | | | | | | |
| 1 | 0330.c6 | No Gate | Lymphocytes | 290 | 778445.30 | 157815.70 | 62.91 | 62.91 |
| | (Sample | | | | | | | |
| | C03.fcs) | | | | | | | |
| 1 | 0330.c6 | No Gate | Monocytes & | 29 | 1444265.36 | 673511.62 | 6.29 | 6.29 |
| | (Sample | | Granulocytes | | | | | |
| | C03.fcs) | | | | | | | |

FIG. 23 and Table 9 show the Accuri particle composition analysis performed before the monocyte sorting experiment, providing information about the composition of the initial monocyte sample in accordance with embodiments of the present document.

TABLE 9

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 45811 | 848578.64 | n/a | 100.00 | 100.00 |
| | (Sample | | | | | | | |
| | D08.fcs) | | | | | | | |
| 1 | 0330.c6 | No Gate | WBC Cells | 36037 | 1306967.97 | 573090.60 | 78.66 | 78.66 |
| | (Sample | | | | | | | |
| | D08.fcs) | | | | | | | |
| 1 | 0330.c6 | No Gate | Stained Cells | 33986 | 1344631.72 | 616567.85 | 74.19 | 74.19 |
| | (Sample | | | | | | | |
| | D08.fcs) | | | | | | | |
| 1 | 0330.c6 | No Gate | Monocytes | 2213 | 1259558.80 | 416870.27 | 4.83 | 4.83 |
| | (Sample | | | | | | | |
| | D08.fcs) | | | | | | | |
| 1 | 0330.c6 | No Gate | Granulocytes & | 31519 | 1349747.33 | 632369.73 | 68.80 | 68.80 |
| | (Sample | | Lymphocytes | | | | | |
| | D08.fcs) | | | | | | | |

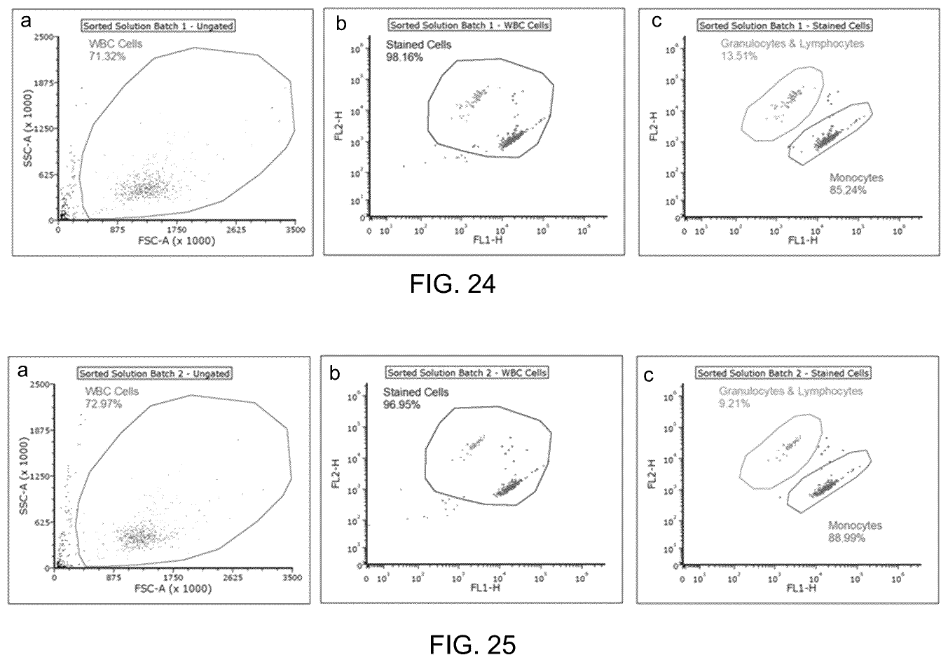

FIG. 24 and Table 10 present the Accuri particle composition analysis for the post-sorting batch 1 solution in the monocyte sorting experiment, offering insights into the composition of the sorted monocytes in accordance with embodiments of the present document.

TABLE 10

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 687 | 669611.93 | 306607.28 | 100.00 | 100.00 |
| | (Sample | | | | | | | |
| | D01.fcs) | | | | | | | |
| 1 | 0330.c6 | No Gate | WBC Cells | 490 | 1301523.26 | 447272.18 | 71.32 | 71.32 |
| | (Sample | | | | | | | |
| | D01.fcs) | | | | | | | |
| 1 | 0330.c6 | No Gate | Stained Cells | 481 | 1315905.51 | 450129.92 | 70.01 | 70.01 |
| | (Sample | | | | | | | |
| | DO1.fcs) | | | | | | | |
| 1 | 0330.c6 | No Gate | Monocytes | 410 | 1299393.82 | 428631.98 | 59.68 | 59.68 |
| | (Sample | | | | | | | |
| | D01.fcs) | | | | | | | |
| 1 | 0330.c6 | No Gate | Granulocytes & | 65 | 1355878.34 | 571474.76 | 9.46 | 9.46 |
| | (Sample | | Lymphocytes | | | | | |
| | D01.fcs) | | | | | | | |

FIG. 25 and Table 11 show the Accuri particle composition analysis for the post-sorting batch 2 solution in the monocyte sorting experiment, providing information about the composition of the sorted monocytes in accordance with embodiments of the present document.

TABLE 11

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 629 | 700115.37 | 328650.42 | 100.00 | 100.00 |
| | (Sample | | | | | | | |
| | D02.fcs) | | | | | | | |
| 1 | 0330.c6 | No Gate | WBC Cells | 459 | 1273964.79 | 436875.96 | 72.97 | 72.97 |
| | (Sample | | | | | | | |
| | D02.fcs) | | | | | | | |
| 1 | 0330.c6 | No Gate | Stained Cells | 445 | 1295238.28 | 437471.45 | 70.75 | 70.75 |
| | (Sample | | | | | | | |
| | D02.fcs) | | | | | | | |
| 1 | 0330.c6 | No Gate | Monocytes | 396 | 1277490.82 | 415947.93 | 62.96 | 62.96 |
| | (Sample | | | | | | | |
| | D02.fcs) | | | | | | | |
| 1 | 0330.c6 | No Gate | Granulocytes & | 41 | 1374305.64 | 632319.16 | 6.52 | 6.52 |
| | (Sample | | Lymphocytes | | | | | |
| | D02.fcs) | | | | | | | |

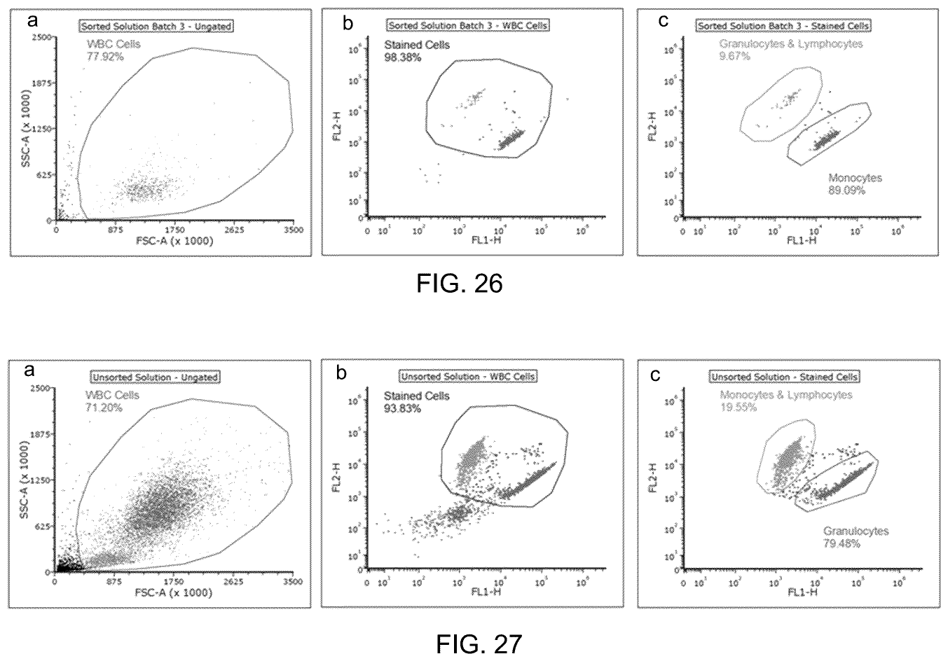

FIG. 26 and Table 12 show the Accuri particle composition analysis for the post-sorting batch 3 solution in the monocyte sorting experiment, offering insights into the composition of the sorted monocytes in accordance with embodiments of the present document.

TABLE 12

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 634 | 783581.20 | 331681.57 | 100.00 | 100.00 |
| | (Sample | | | | | | | |
| | D03.fcs) | | | | | | | |
| 1 | 0330.c6 | No Gate | WBC Cells | 494 | 1293546.96 | 435448.83 | 77.92 | 77.92 |
| | (Sample | | | | | | | |
| | D03.fcs) | | | | | | | |
| 1 | 0330.c6 | No Gate | Stained Cells | 486 | 1308007.69 | 438372.51 | 76.66 | 76.66 |
| | (Sample | | | | | | | |
| | D03.fcs) | | | | | | | |
| 1 | 0330.c6 | No Gate | Monocytes | 433 | 1303526.07 | 418559.13 | 68.30 | 68.30 |
| | (Sample | | | | | | | |
| | D03.fcs) | | | | | | | |
| 1 | 0330.c6 | No Gate | Granulocytes & | 47 | 1253892.77 | 579438.95 | 7.41 | 7.41 |
| | (Sample | | Lymphocytes | | | | | |
| | D03.fcs) | | | | | | | |

FIG. 27 and Table 13 show the Accuri particle composition analysis performed before the granulocyte sorting experiment, providing information about the composition of the initial granulocyte sample in accordance with embodiments of the present document.

TABLE 13

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 10017 | 732781.09 | n/a | 100.00 | 100.00 |
| | (Sample | | | | | | | |
| | A06.fcs) | | | | | | | |
| 1 | 0330.c6 | No Gate | WBC Cells | 7132 | 1338769.27 | 593390.01 | 71.20 | 71.20 |
| | (Sample | | | | | | | |
| | A06.fcs) | | | | | | | |
| 1 | 0330.c6 | No Gate | Stained Cells | 6692 | 1383816.25 | 643393.90 | 66.81 | 66.81 |
| | (Sample | | | | | | | |
| | A06.fcs) | | | | | | | |
| 1 | 0330.c6 | No Gate | Granulocytes | 5319 | 1533598.72 | 831513.89 | 53.10 | 53.10 |
| | (Sample | | | | | | | |
| | A06.fcs) | | | | | | | |
| 1 | 0330.c6 | No Gate | Monocytes & | 1308 | 911936.33 | 229416.51 | 13.06 | 13.06 |
| | (Sample | | Lymphocytes | | | | | |
| | A06.fcs) | | | | | | | |

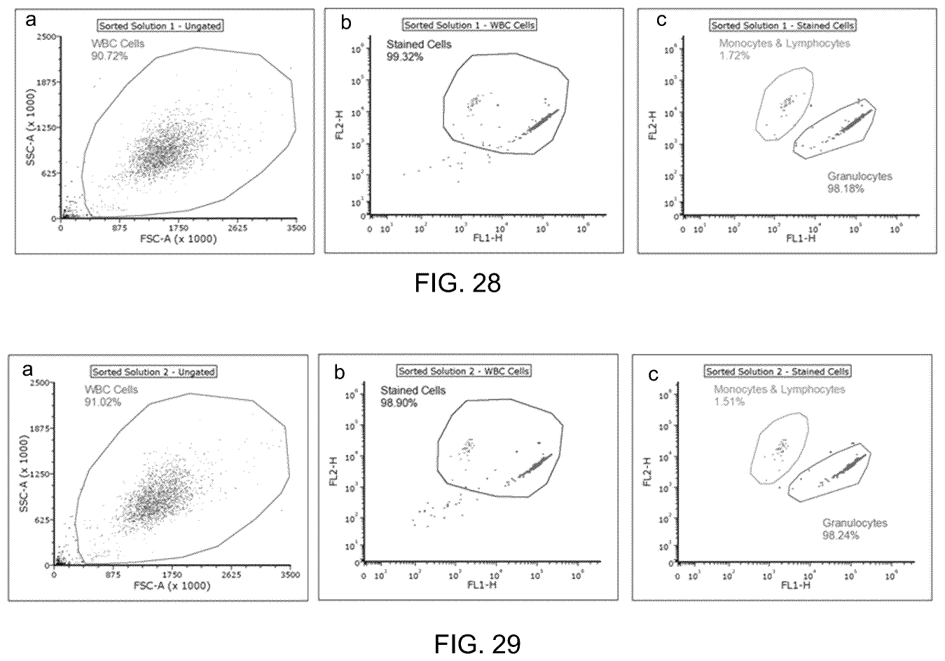

FIG. 28 and Table 14 present the Accuri particle composition analysis for the post-sorting batch 1 solution in the granulocyte sorting experiment, offering insights into the composition of the sorted granulocytes in accordance with embodiments of the present document.

TABLE 14

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 2253 | 1234185.42 | 697154.52 | 100.00 | 100.00 |
| | (Sample | | | | | | | |
| | B01.fcs) | | | | | | | |
| 1 | 0330.c6 | No Gate | WBC Cells | 2044 | 1496009.51 | 862312.82 | 90.72 | 90.72 |
| | (Sample | | | | | | | |
| | B01.fcs) | | | | | | | |
| 1 | 0330.c6 | No Gate | Stained Cells | 2030 | 1502540.62 | 871745.71 | 90.10 | 90.10 |
| | (Sample | | | | | | | |
| | B01.fcs) | | | | | | | |
| 1 | 0330.c6 | No Gate | Granulocytes | 1993 | 1517010.60 | 893306.54 | 88.46 | 88.46 |
| | (Sample | | | | | | | |
| | B01.fcs) | | | | | | | |
| 1 | 0330.c6 | No Gate | Monocytes & | 35 | 856697.18 | 215101.53 | 1.55 | 1.55 |
| | (Sample | | Lymphocytes | | | | | |
| | B01.fcs) | | | | | | | |

FIG. 29 and Table 15 show the Accuri particle composition analysis for the post-sorting batch 2 solution in the granulocyte sorting experiment, providing information about the composition of the sorted granulocytes in accordance with embodiments of the present document.

TABLE 15

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 2206 | 1208918.88 | 677584.67 | 100.00 | 100.00 |
| | (Sample | | | | | | | |
| | B02.fcs) | | | | | | | |
| 1 | 0330.c6 | No Gate | WBC Cells | 2008 | 1488888.55 | 848008.96 | 91.02 | 91.02 |
| | (Sample | | | | | | | |
| | B02.fcs) | | | | | | | |
| 1 | 0330.c6 | No Gate | Stained Cells | 1986 | 1499040.97 | 859358.22 | 90.03 | 90.03 |
| | (Sample | | | | | | | |
| | B02.fcs) | | | | | | | |
| 1 | 0330.c6 | No Gate | Granulocytes | 1951 | 1512725.64 | 878874.68 | 88.44 | 88.44 |
| | (Sample | | | | | | | |
| | B02.fcs) | | | | | | | |
| 1 | 0330.c6 | No Gate | Monocytes & | 30 | 802751.79 | 194681.18 | 1.36 | 1.36 |
| | (Sample | | Lymphocytes | | | | | |
| | B02.fcs) | | | | | | | |

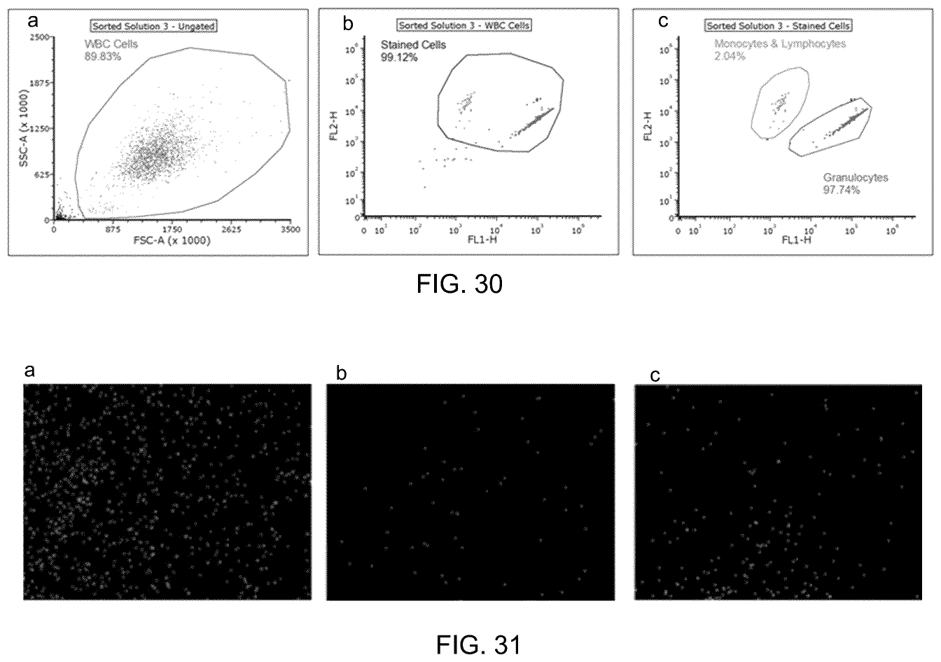

FIG. 30 and Table 16 show the Accuri particle composition analysis for the post-sorting batch 3 solution in the granulocyte sorting experiment, offering insights into the composition of the sorted granulocytes in accordance with embodiments of the present document.

TABLE 16

| | | | | | X | Y | % of | % of |
|---|---|---|---|---|---|---|---|---|
| Overlay | | Overlay | | # of | Geometric | Geometric | gated | all |
| # | Filename | Gate | Gate | Events | Mean | Mean | cells | cells |
| 1 | 0330.c6 | No Gate | None | 2035 | 1174951.85 | 646446.96 | 100.00 | 100.00 |
| | (Sample | | | | | | | |
| | B03.fcs) | | | | | | | |
| 1 | 0330.c6 | No Gate | WBC Cells | 1828 | 1461223.56 | 829439.29 | 89.83 | 89.83 |
| | (Sample | | | | | | | |
| | B03.fcs) | | | | | | | |
| 1 | 0330.c6 | No Gate | Stained Cells | 1812 | 1469872.83 | 837973.10 | 89.04 | 89.04 |
| | (Sample | | | | | | | |
| | B03.fcs) | | | | | | | |
| 1 | 0330.c6 | No Gate | Granulocytes | 1771 | 1488778.29 | 866970.93 | 87.03 | 87.03 |
| | (Sample | | | | | | | |
| | B03.fcs) | | | | | | | |
| 1 | 0330.c6 | No Gate | Monocytes & | 37 | 781934.67 | 166146.68 | 1.82 | 1.82 |
| | (Sample | | Lymphocytes | | | | | |
| | B03.fcs) | | | | | | | |

In some embodiments, the system incorporates various sample preparation techniques to assess the system's capabilities in imaging and sorting fluorescent polystyrene particles, CHO-ES cells, MCF7 cells, human iPSC (induced pluripotent stem cells), and human white blood cells.

Fluorescent Polystyrene Particles Preparation The system evaluates the imaging and sorting performance of the low-latency IACS, utilizing fluorescent PS beads. A 1:6 mixture of 15 μm PS particles (Fluorescent microspheres, Dragon Green, Cat. No. FSDG009, Bangs Laboratories, Inc.) and 7 μm PS particles (Fluorescent microspheres, Dragon Green, Cat. No. FSDG007, Bangs Laboratories, Inc.) is introduced from the sample inlet of a microfluidic chip. The system adjusts the concentration of these particles to 500 particles μL−1.

CHO-ES Cells and DNA Staining Preparation The system uses CHO-K1 cells (ATCC CCL-61) for DNA staining. The cells are harvested at a confluency of approximately 80%. The harvested cells undergo centrifugation at 350×g for 5 minutes, the supernatant is removed and the cells are washed with PBS (Genesee Scientific, CA, USA). This washing process is repeated, after which 100 μL of 4% formaldehyde, methanol-free (Cell Signaling Technology, Massachusetts, USA) is added per million cells. Following incubation for 15 minutes at 37° C., the fixed cells are washed and resuspended in PBS containing 0.5% BSA (Thermo Scientific) at a concentration of 1.0×106 cells/mL. Lastly, the cells are stained with 0.5 μM Vybrant DyeCycle Green Stain (Invitrogen) for 30 minutes and filtered using a 35 μm strainer cap (Genesee Scientific, CA, USA).

MCF7 Cells and Mitochondrial Staining Preparation MCF7 cells (ATCC HTB-22) are prepared for imaging their mitochondria. The cells, harvested at a confluency of 70%, are diluted to a concentration of 1.0×106 cells/mL using a buffer composed of PBS, 0.5% BSA, 12.5 mM HEPES (Gibco), and 5 mM EDTA (Invitrogen). The diluted cells are stained with 100 mM of Mito View Green (Biotium, San Francisco, USA) and incubated for 15 minutes at 37° C. Post incubation, the cells are filtered with a 35 μm stainer cap and analyzed.

Human iPSC Cells and Viability Staining Preparation Human iPSCs reprogrammed from fibroblasts are cultured in DMEM/F-12 50/50 1× (Corning™, #10-092-CM) supplemented with HiDef B8 500X (Defined Bioscience, #LSS-201). Non-TC treated 6-well plates (CELLTREAT, #229506) are treated with vitronectin (Gibco™, #A14700), a recombinant human protein that provides a defined surface for feeder-free culture. Samples are maintained with a visual assessment of less than 30% differentiation per well. Cells are passaged in aggregates ranging from 50-100 μm, using the enzyme-free Gentle Cell Dissociation Reagent (STEMCELL Technologies, #100-0485). Healthy iPSC colonies are identified by morphology under phase microscopy for colony compactness, defined borders, well-outlined edges, and a large nucleus-to-cytoplasm ratio. A single-cell suspension is obtained using Accutase® (Innovative Cell Technologies, Inc. #AT104), centrifuged at 200×g for 3 minutes, and resuspended in sheath buffer (basal media+10% Accutase) at a concentration of 3.0×105 cells/mL. Live calcein AM (Invitrogen™, #C3099) stained iPSCs are imaged by capturing conversion of the green, fluorescent calcein (Ex/Em: 494/517 nm).

Human White Blood Cells and Immune Staining Preparation The system employs the Veri-Cells™ Leukocyte Kit, prepared from lyophilized human peripheral blood leukocytes (BioLegend Cat. 426003). These cells work with commonly tested cell surface markers such as CD3, CD14, CD19, and CD66b. CD66b is a glycosylphosphatidylinositol (GPI) linked protein expressed on granulocytes, CD3 and CD19 are expressed on T-cell and B-cell, respectively, and CD14 is expressed at high levels on monocytes. The system uses various combinations of specific antibodies listed in Supplementary Table 2 for leukocyte phenotyping. The concentration of the particles is adjusted to be between 500 and 1000 particles μL−1 to achieve an event rate of approximately 100-200 events per second (eps).

The described system for image acquisition and sorting provides a comprehensive approach for sample preparation in various experiments. The system demonstrates its capability to handle and analyze different types of samples, including fluorescent particles, cells stained with specific dyes, and immune-stained human blood cells. This enables the evaluation of the system's performance in imaging and sorting diverse biological samples, showcasing its versatility and potential applications in the field.

### EXAMPLES

The following examples are illustrative of several embodiments in accordance with the present technology. Other exemplary embodiments of the present technology may be presented prior to the following listed examples, or after the following listed examples.

In some embodiments in accordance with the present technology (example A1), a particle flow device including a substrate, a channel formed on the substrate operable to allow individual particles to flow along a flow direction to a first region of the channel, and two or more output paths branching from the channel at a second region proximate to the first region in the channel, an imaging system interfaced with the particle flow device and operable to obtain image data associated with a particle when the particle is in the first region during flow through the channel, a control command unit including a processor configured to produce a control command indicative of a particle class determined based on a gating model and the image data; and an actuator operatively coupled to the particle flow device and in communication with the control command unit, the actuator being operable to direct the particle into an output path of the two or more output paths based on the control command, wherein the image-activated particle sorting system is operable to sort the individual particles during flow in the channel.

Example A2 includes the system of any one of examples herein, in which the control command is produced when the particle is flowing through the channel.

Example A3 includes the system of any one of examples herein, in which a latency from a first time of image capture of the particle to a second time of the particle being directed by the actuator is within a time frame of 15 milliseconds or less. For example, the latency is less than 10 milliseconds, 8 milliseconds, 6 milliseconds, 5 milliseconds, or 3 milliseconds.

Example A4 includes the system of any one of examples herein, in which the gating model is a machine learning model trained to predict the particle's class based on the image data.

Example A5 includes the system of any one of examples herein, in which the gating model includes a convolutional neural network (CNN) based Artificial Intelligence (AI) model.

Example A6 includes the system of any one of examples herein, in which a kernel count of initial convolutional kernels of the AI model is lower than 10 such that a training time to train the gating model using the processor of the control command unit is no more than 2 hours and a classification accuracy of the gating model for determining particle classes of the individual particles is at least 90%.

Example A7 includes the system of any one of examples herein, in which the individual particles are label-free, the imaging system is configured to obtain transmission images of the individual particles, and the control command unit is configured to generate control commands for the individual particles based on the gating model and the corresponding transmission images.

Example A8 includes the system of any one of examples herein, in which the imaging system includes one or more light sources to provide an input light to the first region of the particle flow device, and an optical imager to capture the image data from the particles illuminated by the input light in the first region.

Example A9 includes the system of any one of examples herein, in which the one or more light sources include at least one of a laser or a light emitting diode (LED).

Example A10 includes the system of any one of examples herein, in which the optical imager includes an objective lens optically coupled to a spatial filter, an emission filter, and a photomultiplier tube.

Example A11 includes the system of any one of examples herein, in which the optical imager further includes one or more light guide elements to direct the input light to the first region, to direct light emitted or scattered by the particle to an optical element of the optical imager, or both.

Example A12 includes the system of any one of examples herein, in which the light guide element includes a dichroic mirror.

Example A13 includes the system of any one of examples herein, in which the optical imager includes two or more photomultiplier tubes to generate two or more corresponding signals based on two or more bands or types of light emitted or scattered by the particle.

Example A14 includes the system of any one of examples herein, in which the imaging system includes a digitizer configured to obtain the image data that includes time domain signal data associated with the particle imaged in the first region on the particle flow device.

Example A15 includes the system of any one of examples herein, in which a data processing unit is in communication with the imaging system and the control command unit, the data processing unit being configured to process the image data obtained by the imaging system and output a particle image for the particle to be used as input to the gating model.

Example A16 includes the system of any one of examples herein, in which the control command unit comprises a first processor, and the data processing unit comprises a second processor that is different from the first processor.

Example A17 includes the system of any one of examples herein, in which the first processor comprises a graphics processing unit (GPU); and the second processor comprises a field-programmable gate-array (FPGA).

Example A18 includes the system of any one of examples herein, in which the particle flow device includes a microfluidic device or a flow cell integrated with the actuator on the substrate of the microfluidic device or the flow cell.

Example A19 includes the system of any one of examples herein, in which the actuator includes a piezoelectric actuator coupled to the substrate and operable to produce a deflection to cause the particle to move in a direction that directs the particle along a trajectory to the output path of the two or more output paths.

Example A20 includes the system of any one of examples herein, in which the particles include cells, and the one or more properties associated with a cell includes an amount or a size of a features of or on the cell, one or more sub-particles attached to the cell, or a particular morphology of the cell or portion of the cell.

Example A21 includes the system of any one of examples herein, in which the particles include cells, and the sorting criteria includes a cell contour, a cell size, a cell shape, a nucleus size, a nucleus shape, a fluorescent pattern, or a fluorescent color distribution.

Example A22 includes the system of any one of examples herein, in which the particles include cells, and the one or more properties associated with the cell includes a physiological property of the cell including a cell life cycle phase, an expression or localization of a protein by the cell, a damage to the cell, or an engulfment of a substance or sub-particle by the cell.

In some embodiments in accordance with the present technology (example A23), a method for image-based sorting of a particle includes obtaining, by an imaging system interfaced with a particle flow device, image data of a particle flowing through a channel of the particle flow device; producing, by a control command unit, a control command indicative of a particle class of the particle determined based on a gating model and the image data; and directing the particle into one of a plurality of output paths of the particle flow device based on the control command.

Example A24 includes the method of any one of examples herein, in which the control command is produced when the particle flows through the channel.

Example A25 includes the method of any one of examples herein, in which the gating model is a machine learning model trained to predict the particle class based on the image data.

Example A26 includes the method of any one of examples herein, in which the method includes allowing individual particles to flow through the channel; obtaining, by the imaging system, imaging data of the individual particles during flow through the channel; producing, by the control command unit, control commands indicative of particle classes of the individual particles that are determined based on the gating model and the image data of the individual particles while the individual particles flow through the channel; and directing the individual particles into the plurality of output paths of the particle flow device according to the control commands.

Example A27 includes the method of any one of examples herein, in which a latency between image capture of the particle and actuation of an actuator to direct the particle is within a time frame of 15 milliseconds or less. For example, the latency is less than 10 milliseconds, 8 milliseconds, 6 milliseconds, 5 milliseconds, or 3 milliseconds.

Example A27 includes the method of any one of examples herein, in which the gating model comprises a convolutional neural network (CNN) based Artificial Intelligence (AI) model.

Example A28 includes the method of any one of examples herein, in which the method further includes obtaining transmission images of the individual particles; and generating control commands for the individual particles based on the gating model and the corresponding transmission images.

In some embodiments in accordance with the present technology (example B1), a system includes a particle flow device structured to include a substrate, a channel formed on the substrate operable to flow individual cells along a flow direction to a first region of the channel, and two or more output paths branching from the channel at a second region proximate to the first region in the channel, an imaging system interfaced with the particle flow device and operable to obtain image data associated with a cell when the cell is in the first region during flow through the channel, a control command unit including a processor configured to produce a control command indicative of a cell class determined based on a gating model and the image data; and an actuator operatively coupled to the particle flow device and in communication with the control command unit, the actuator being operable to direct the cell into an output path of the two or more output paths based on to the control command, wherein the system is operable to sort the individual cells during flow in the channel.

In some embodiments in accordance with the present technology (example B2), a method for image-based sorting of a cell includes obtaining, by an imaging system interfaced with a particle flow device, image data of a cell flowing through a channel of the particle flow device; producing, by a control command unit, a control command indicative of a cell class of the cell determined based on a gating model and the image data; and directing the cell into one of a plurality of output paths of the particle flow device based on the control command.

In some embodiments in accordance with the present technology (example C1), a real-time image-activated particle sorting microfluidic system includes a cell sorting system including a microfluidic channel configured to allow one or more particles to flow therein in a first direction; an imaging unit including one or more lenses and an imaging detector operable to obtain image data as the one or more particles are flowing in the microfluidic channel; a processor including, or coupled to, an artificial intelligence system coupled to the imaging unit to receive the image data and to determine a class of the one or more particles; and a transducer coupled to the processor and to the cell sorting system, wherein upon determination that a first of the one or more particles is classified as having a particular particle class, the processor is configured to provide a signal to actuate the transducer to direct the first of the one or more particles to a first output of the microfluidic channel.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Various embodiments described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), Blu-ray Discs, etc. Therefore, the computer-readable media described in the present application include non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

For example, one aspect of the disclosed embodiments relates to a computer program product that is embodied on a non-transitory computer readable medium. The computer program product includes program code for carrying out any one or and/or all of the operations of the disclosed embodiments.

Only a few implementations and examples are described and other implementations, enhancements and variations can be made based on what is described and illustrated in this patent document.

1. (canceled)

2. An image-activated particle sorting system, comprising:
a particle flow device comprising (i) a substrate and a channel formed on the substrate and configured to allow individual particles to flow through the channel along a flow direction to a first region of the channel, and (ii) two or more output paths branching from the channel at a second region proximate to the first region in the channel;
an imaging system interfaced with the particle flow device and configured to obtain image data associated with a particle flowing in the first region through the channel;
a control command unit comprising a processor configured to produce a control command indicative of a particle class determined based at least in part on a gating model and the image data, wherein: (i) the control command is produced when the particle is flowing through the channel, and (ii) the gating model comprises a machine learning model trained to predict the particle class based at least in part on the image data; and
an actuator operatively coupled to the particle flow device and in communication with the control command unit configured to direct the particle into an output path of the two or more output paths based at least in part on the control command and thereby sort the individual particles during flow in the channel.

3. The system of claim 2, wherein the image-activated particle sorting system has a latency of less than about 15 milliseconds, wherein the latency is measured from (i) a first time point at which the image data is obtained by the imaging system to (ii) a second time point at which the particle is being directed by the actuator into the output path.

4. The system of claim 2, wherein the gating model comprises a convolutional neural network (CNN) based Artificial Intelligence (AI) model.

5. The system of claim 4, wherein a kernel count of initial convolutional kernels of the AI model is lower than 10 such that a training time to train the gating model using the processor of the control command unit is no more than 2 hours.

6. The system of claim 4, wherein a kernel count of initial convolutional kernels of the AI model is lower than 10 such that a classification accuracy of the gating model for determining particle classes of the individual particles is at least 90%.

7. The system of claim 4, wherein the CNN comprises a UNet model.

8. The system of claim 7, wherein the UNet model is optimized to reduce an initial kernel count of initial convolution kernels in the UNet model.

9. The system of claim 8, wherein the UNet model is optimized to reduce an initial kernel count of initial convolution kernels for reducing a model parameter, a model size, a training time reduction, and an inference time.

10. The system of claim 2, wherein:
the individual particles are label-free,
the imaging system is configured to obtain transmission images of the individual particles, and
the control command unit is configured to generate control commands for the individual particles based at least in part on the gating model and corresponding transmission images.

11. The system of claim 2, wherein the imaging system comprises one or more light sources configured to provide an input light to the first region of the particle flow device, and an optical imager configured to capture imaging data from the individual particles illuminated by the input light in the first region.

12. The system of claim 11, wherein the one or more light sources comprise at least one of a laser or a light emitting diode (LED).

13. The system of claim 11, wherein the optical imager comprises an objective lens optically coupled to at least one of a band-pass optical filter or a photomultiplier tube.

14. The system of claim 13, wherein the optical imager further comprises one or more light guide elements configured to direct the input light to the first region, to direct light emitted or scattered by the individual particles to an optical element of the optical imager, or both.

15. The system of claim 11, wherein the optical imager comprises two or more photomultiplier tubes configured to generate two or more corresponding signals based at least in part on two or more bands or types of light emitted or scattered by the individual particles.

16. The system of claim 11, wherein the imaging system comprises a digitizer configured to obtain the image data that comprises time domain signal data associated with the particle imaged in the first region on the particle flow device.

17. The system of claim 2, further comprising:
a data processing unit in communication with the imaging system and the control command unit, wherein the data processing unit is configured to process the image data obtained by the imaging system and output a particle image for the particle to be used as input to the gating model.

18. The system of claim 17, wherein the control command unit comprises a first processor and the data processing unit comprises a second processor, wherein the second processor is different from the first processor.

19. The system of claim 2, wherein the particle flow device comprises a microfluidic device or a flow cell integrated with the actuator on the substrate of the microfluidic device or the flow cell.

20. The system of claim 2, wherein the actuator comprises a piezoelectric actuator coupled to the substrate and configured to produce a deflection to cause the particle to move in a direction that directs the particle along a trajectory to the output path of the two or more output paths.

21. The system of claim 2, wherein the system further comprises a digitizer or a digital signal processing (DSP) module, wherein the digitizer is configured to capture the image data of the individual particles, and wherein the DSP module is configured to reconstruct a particle image via a temporal-spatial transformation.
